(19) United States
(12) Patent Application Publication
(10) Pub. No.: US 2024/0263252 A1
(43) Pub. Date: Aug. 8, 2024

(54) USING DEEP RNA SEQUENCING DATA TO IDENTIFY ANTIMICROBIAL-RESISTANT BACTERIA

(71) Applicant: Rhode Island Hospital, Providence, RI (US)

(72) Inventors: Sean F. Monaghan, East Greenwich, RI (US); Gerard J. Nau, Sharon, MA (US)

(21) Appl. No.: 18/528,750

(22) Filed: Dec. 4, 2023

Related U.S. Application Data
(60) Provisional application No. 63/378,365, filed on Oct. 4, 2022.
(60) Provisional application No. 63/378,366, filed on Oct. 4, 2022.

(51) Int. Cl. C12Q 1/689; C12Q 1/686

(52) U.S. Cl. CPC C12Q 1/689; C12Q 1/686; C12Q 2600/158

(57) ABSTRACT

Blood and lung infections are a worldwide health problem with approximately five million deaths each year. Early treatment could help save lives, but better tests to identify these infections are needed. This invention identifies pathogens, particularly those that are resistant to antibiotics, faster. This invention allows for appropriate antibiotic treatment at an earlier time point. Data from deep RNA sequencing of human blood is used to create a faster diagnostic test for infections and associated antimicrobial resistance.

### CROSS-REFERENCE TO RELATED APPLICATIONS

This patent application claims priority under 35 U.S.C. § 119(e) to the provisional patent applications U.S. Ser. No. 63/378,365 and U.S. Ser. No. 63/378,366, both filed Oct. 4, 2022.

### STATEMENT REGARDING FEDERALLY SPONSORED RESEARCH OR DEVELOPMENT

This invention was made with government support under P20 GM121344, R35 GM118097, R01 GM127472, and R35 GM142638 awarded by the National Institutes of Health. The government has certain rights in the invention.

### TECHNICAL FIELD OF THE INVENTION

This invention generally relates to the chemical analysis of biological material, using nucleic acid products used in the analysis of nucleic acids, e.g., primers or probes for diseases caused by alterations of genetic material.

### BACKGROUND OF THE INVENTION

Antimicrobial-resistant bacteria cause almost five million deaths each year. Global burden of bacterial antimicrobial resistance in 2019: A systematic analysis (2022). Early identification of the causative pathogen and its antibiotic resistance pattern is central to infection management by focusing on antimicrobial administration. Typical clinical practice in patients with infections would benefit by starting treatment with broad-spectrum antibiotics as early as possible.

Despite the benefit of broad-spectrum antibiotics in reducing infection mortality, there are also negative consequences. Broad-spectrum antibiotics are costly and labor-intensive. They increase the risk of Clostridioides difficile colitis and select new, antibiotic-resistant pathogens.

Culturing the site of infection identifies the pathogen and its associated antibiotic resistance but can take days to generate actionable information. Antibiotics administered before sample acquisition can reduce culture yields.

There is a need in the biomedical art for diagnostic tests for diagnosing and treating sepsis.

### SUMMARY OF THE INVENTION

The invention provides molecular diagnostic tests to overcome the limitations of conventional microbiological approaches for diagnosing and treating sepsis.

In a first embodiment and as a proof of principle, the inventors develop polymerase chain reaction (PCR) diagnostic tests for four common bacteria: Staphylococcus aureus, extra-intestinal Escherichia coli, Pseudomonas aeruginosa, and Haemophilus influenzae.

In a second embodiment, the inventors create tests of clinically relevant resistance genes associated with these four pathogens. These bacteria are pathogens of interest in the request for applications (RFA-AI-22-010) for bacteremia (S. aureus, E. coli, P. aeruginosa) and pneumonia (S. aureus, P. aeruginosa, H. influenzae). These pathogens are also the most common causes of bacteremia and hospital-acquired pneumonia at Rhode Island Hospital. These four pathogens have antimicrobial resistance attributable to specific genes requiring antibiotic management changes. These four pathogens are the top organisms that cause death due to resistance. Global burden of bacterial antimicrobial resistance in 2019 (2022).

In a third embodiment, the invention provides tests of clinically relevant resistance genes associated with other pathogens that cause sepsis.

In a fourth embodiment, the invention provides a diagnostic PCR test based on bacterial RNA. PCR tests were developed for respiratory pathogens. See Covert, Bashore, Edds, & Lewis (2021). PCR tests were developed for identifying the DNA of bacteria like S. aureus in targeted sites. Palavecino (2020). Pathogen identification was previously done by sequencing cell-free DNA from blood. Camargo et al. (2019).

In the diagnostic PCR test, the most abundant RNA targets are selected from the blood of patients with these infections, making the approach more sensitive than single-copy DNA targets. Antibiotic resistance correlates closely with gene expression. The risk of RNA degradation is significantly mitigated by stabilizing RNA. Because the targets are derived from RNA sequencing data, those RNAs are abundant and measurable in patients with infection.

In one aspect, the unmapped RNA reads from patients with infections that align with pathogens can inform a better diagnostic test. PCR targets for diagnostics come from a data set created from deep sequencing (>100 million reads) of the blood of patients with bacteremia or pneumonia. Pathogen identification is performed with standard culture techniques. The RNA sequences from the pathogens are typically discarded in transcription analysis because they would not align with the human genome. These “unmapped reads” are being identified in the blood of patients and aligned to a custom “genome” derived from them pathogens of interest to identify the causative organism. RNAs that align with resistance genes are also identified.

In a fifth embodiment (A1a), the invention provides a direct from blood, without culture, reverse transcriptase polymerase chain reaction (RT-qPCR) test for bacteria causing bacteremia, specifically S. aureus, E. coli, P. aeruginosa, based on the RNA identified in patients with bacteremia caused by these organisms.

In a sixth embodiment (A1b), the invention provides a method to validate these RT-qPCR tests in samples from patients with and without bacteremia.

In a seventh embodiment (A2a), the invention provides a direct from blood, without culture, reverse transcriptase polymerase chain reaction (RT-qPCR) test for bacteria causing pneumonia, specifically S. aureus, P. aeruginosa, H. influenzae, based on the RNA identified in patients with pneumonia caused by these organisms.

In an eighth embodiment (A2b), the invention provides a method to validate these RT-qPCR tests in samples from patients with and without pneumonia.

In an eighth embodiment (A3a), using the RNA from patients with infections, the invention provides an RT-PCR for the most common resistance genes that would influence treatment for S. aureus, E. coli, P. aeruginosa, and H. influenzae.

In a ninth embodiment (A3b), the invention provides a method to validate these PCR tests for resistance genes in samples from patients with and without infections.

In a tenth embodiment, the invention provides a direct from blood RT-PCR test for bacteremia caused by S. aureus, E. coli, and P. aeruginosa without culture with phenotypic microbial resistance identification.

In an eleventh embodiment, the invention provides a direct from blood RT-PCR test for pneumonia caused by S. aureus, P. aeruginosa, and H. influenzae without culture with phenotypic microbial resistance identification.

In a twelfth embodiment, the invention provides that all tests can have a result in fewer than four hours from the time of sample collection.

In a thirteenth embodiment, the invention provides the ability to standardize and scale these tests for a clinical microbiology setting. See TABLE 3 below.

In another aspect, the invention provides a direct form blood PCR panel (e.g., using the top twelve pathogens) that identifies the pathogens and resistance profile faster (fewer than four hours) than current bacteremia and hospital-acquired pneumonia techniques. This invention translates deep RNA sequencing data into a product: a rapid PCR to identify S. aureus, E. coli, P. aeruginosa, and H. influenza and potential resistance genes without culture or specimens other than blood.

Several factors have recently provided improvements to RNA sequencing that can make RNA sequencing useful for diagnostic and therapeutic methods of treating sepsis using a standard set of testing conditions. These improvements are supported by the literature and preliminary data indicating that RNA sequencing can identify bacterial pathogens directly from the blood of patients with those infections.

An Illumina Machine (NovaSeq X+) should be able to take only thirteen hours to obtain 1.6 billion reads, not including processing time.

The commonly identified and organism-specific sequences (for S. aureus, E. coli, P. aeruginosa, and H. influenza) are the template for designing oligonucleotide primers for RT-qPCR tests. Future efforts to expand the diagnostic test to other pathogens may require RNA sequencing from patients with pneumonia due to other pathogens.

Direct RNA sequencing should be done for these assays. This conversion of RNA to DNA takes time. Direct RNA sequencing allows faster processing times, to be completed in the 4-hour time frame. Focusing on RNA rather than DNA improves phenotypic correlation with antimicrobial resistance.

RNA sequencing can be a valuable tool in personalizing the care of sepsis patients. With these advances, this tool will be used by clinicians in the Intensive Care Unit caring for sepsis patients. The improvements described above should expand the technology from the research laboratory to the clinical microbiology laboratory.

The improvements listed enable a direct from blood, without culture, reverse transcriptase polymerase chain reaction (RT-qPCR) test for bacteria and methods to validate these RT-qPCR tests in samples from patients. RT-qPCR tests are useful in the diagnosis and treatment of sepsis. The improvements listed above also enable methods for combatting the scourge of drug-resistant bacteria.

The improvements listed are useful for designing better platforms and reagents for direct from blood, without culture, reverse transcriptase polymerase chain reaction (RT-qPCR) tests for bacteria. QIAGEN (Germantown, MD, USA) is a manufacturer of platforms and reagents for RNA isolation and sequencing. Abbott (Abbott Park, IL, USA), Cepheid (Sunnyvale, CA, USA), Thermo Fisher Scientific (Waltham, MA, USA), and ELITech Group (Puteaux, FR) are also manufacturer of platforms and reagents for RNA isolation and sequencing.

RNA sequencing can be a medically useful tool for personalizing the care of sepsis patients. With these advances, this tool will be used by clinicians in the Intensive Care Unit caring for sepsis patients.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 shows the RT-qPCR validation of sequencing results. cDNA from patient RNA was tested for the SARS-COV-2 N gene using real-time PCR. FIG. 1A is a map of the N gene showing the location of the peak of sequencing reads (red box) and primers directed to the peak or elsewhere in the gene (“off peak). FIG. 1B is a bar graph showing the relative expression level of peak and off-peak sequences in fifteen COVID-19 patients. The peak sequence is shown in red and off-peak in black.

FIG. 2A is a schematic showing the structure of a SARS-COV2-Nucleocapsid (N2) gene. FIG. 2B is a bar chart showing. FIG. 2C is a bar chart showing.

FIG. 3 is a schematic showing significant differences of the minimum free energy of RNA-Seq reads in different genes. Lines between the N gene and ORF1ab, between N gene and ORF6, and between ORF6 and the S gene represent the significant differences between genes.

FIG. 4 is a schematic showing significant differences of the ensemble free energy of RNA-Seq reads in different genes. Lines between the N gene and ORF1ab, between N gene and ORF6, between ORF6 and the S gene, and between ORF6 and ORF3a represent the significant differences between genes.

FIGS. 5A-5C are a set of bar graphs showing examples of motif effects on energy. FIG. 5A shows the results for minimum free energy (left) and ensample free energy (right) for samples that have the motif MEME-ChIP 9 compared with samples that do not. The MEME-ChIP 9 motif may increase the minimum free energy (see left). This motif may destabilize RNA sequence reads and may increase the ensemble free energy (see right). FIG. 5B shows the results for minimum free energy (left) and ensample free energy (right) for samples that have the motif MEME-28 compared with samples that do not. The MEME-28 motif may decrease the minimum free energy (see left). This motif may stabilize RNA sequence reads (see right) and decrease the ensemble free energy.

FIG. 5C shows the results for minimum free energy (left) and ensample free energy (right) for samples that have the motif MEME-15 compared with samples that do not. The MEME-15 motif does not seem to affect the minimum free energy (see left). This motif may not affect RNA sequence read stability (see right) and does not seem to affect the ensemble free energy.

FIG. 6 is a set of calculated sequence results from MEME-ChIP analysis. Only ten results were requested to be given with three significantly affecting the minimum free energy and ensemble free energy of read sequences.

FIG. 7 is a set of exemplary secondary structures of reads based on minimum free energy with discovered motifs. FIG. 7A shows MEME-ChIP 8. FIG. 7B shows MEME-ChIP 9. FIG. 7C shows MEME-ChIP 10. FIG. 7D shows MEME-9. FIG. 7E. MEME-10. FIG. 7F. MEME-21. FIG. 7G. MEME-22. FIG. 7H. MEME-27. FIG. 7I. MEME-28.

FIG. 8 is a chart of density plots used to identify the areas of the genomes with the most reads.

FIG. 9 is a pair of reads from two groups of patients with positive blood cultures compared to patients that were found to not have an E. coli infection.

### DETAILED DESCRIPTION OF THE INVENTION

### Industrial Applicability

Faster pathogen identification for severe infections. Sepsis causes one out of five deaths in the world. Rudd et al. (2020). The diagnosis of septic infection is a significant challenge for sepsis care. Duncan, Youngstein, Kirrane, & Lonsdale (2021). This invention provides better diagnoses of bacterial infections and associated antimicrobial resistance to improve outcomes.

The Surviving Sepsis Campaign standardized treatment for sepsis that includes blood cultures before broad-spectrum antibiotics and start of antibiotics within one hour. See Evans et al. (2021). In a multivariate analysis of factors affecting mortality in patients with septic shock, the time to begin antibiotic treatment was the most impactful variable. Kumar et al. showed this impact, reporting a 79.9% survival in septic shock patients with antibiotics in the first hour and a reduction of 7.6% for every hour delay. Kumar et al. (2006). Vazquez-Guillamet et al. determined that the number needed to treat with antibiotics to save one life was five. Vazquez-Guillamet et al., (2014). Faster pathogen identification improves sepsis outcomes by guiding antibiotic selection. Current methods take too much time, such as days. Sepsis kills in hours.

This invention provides diagnostic tests for three pathogens that cause bacteremia and three pathogens that cause pneumonia to shorten the time to pathogen-specific treatment for diseases such as sepsis.

Antimicrobial resistance is a significant health problem. Across the world, antimicrobial-resistant bacteria cause almost five million deaths each year. Global burden of bacterial antimicrobial resistance in 2019: A systematic analysis (2022 Antimicrobial-resistant bacteria cause over 100,000 deaths in the United States with costs of over $21 billion. With each antibiotic, resistance follows shortly after that. See Clatworthy, Pierson, & Hung (2007). The United Nations convened a high-level meeting on Antimicrobial Resistance on Sep. 21, 2016, that included statements by global leaders such as Secretary-General Ban Ki-moon: “Drug resistance imposes huge costs on health systems and is taking a growing—and unnecessary—toll in lives and threatening to roll back much of the progress we have made.” Locally the trend is increased in resistance also increasing among many pathogens. Kassakian & Mermel (2014).

Importance of phenotypic antibacterial susceptibility. Bacteria have multiple antimicrobial resistance mechanisms and are easily transferred, resulting in pathogens with extensive resistance profiles. Harbottle, Thakur, Zhao, & White (2006). Despite advances in genomic testing, resistance genes found in DNA do not always correlate with phenotypic resistance. Bortolaia et al. (2020). Some computational approaches were suggested to handle this data. Bortolaia et al. (2020). Reasons for the lack of correlation between genomic data and resistance phenotypes include lack of transcription and DNA not associated with a living cell. Using RNA sequencing data allows for a better correlation between the genomic data and the expression of resistance. Using RNA data identifies only genes actively being transcribed, thus measuring gene expression levels.

Facilitate antimicrobial stewardship. Antibiotic stewardship was suggested as a method to combat resistance. Broad-spectrum antibiotics, although appropriate, have an adjusted increased mortality risk. Webb et al. (2019). Other studies specifically state that broad-spectrum antibiotics increase the risk of in-hospital death when no resistance is identified. Rhee et al. (2020). Broad-spectrum coverage is used in 67.8% of patients. Rhee et al. (2020). De-escalation is important because new resistance emerges each day of inappropriate antibiotic exposure. Teshome et al. (2019). In the hospital setting, empiric therapy is only de-escalated 16% of the time. De Bus et al. (2020). Costs are reduced when de-escalation is used. Seok, Jeon, & Park (2020). Narrowing antibiotics reduces labor in the ICU. Mei-Sheng, Riley & Olans (2021). A diagnostic test that rapidly identifies a pathogen and its antimicrobial resistance should help in antimicrobial stewardship.

In the twelfth embodiment above, PCRs informed by a large dataset are performed in four hours, yielding direct from blood results independent of culture. Al-Hasan, Winders, Bookstaver, & Justo recommend directly assessing stewardship programs rather than looking for adverse events. Al-Hasan, Winders, Bookstaver, & Justo (2019). With faster bacteria identification, serial testing could assess treatment efficacy. Serial testing could be an additional metric in stewardship programs as antibiotics could be stopped sooner.

Unmapped reads identify bacterial RNA with deep RNA sequencing. In the initial assessment of RNA sequencing data, the reads are aligned to the genome of the species the sample came from, commonly the human genome. Unmapped reads can account for up to 20% of the data. These data are typically discarded. In the samples of humans with the illness, there are more unmapped reads (˜35%). Monaghan et al. (2021). The Read Origin Protocol (ROP) (Mangul et al. (2018)) and Kraken (Wood, Lu, & Langmead (2018)) have been developed to determine the origin of unmapped reads. The Read Origin Protocol analysis of multiple data sets mapped 99.9% of all reads. The data typically discarded were analyzed in a seven-step process. One step is of particular interest because of the relevance to the patient population in this work: bacterial reads. Using ROP, or more recently Kraken2, bacterial RNA was identified in the blood samples of patients with sepsis, which mapped to the bacteria found in blood culture. RNA sequencing data can inform primer design to produce better diagnostic tests.

Diagnostic solution. This invention leverages a large data set of unmapped reads from patients diagnosed with infections by the gold standard of bacterial culture. This deep RNA sequencing data suggest PCR primers to identify pathogens, such as S. aureus, E. coli, P. aeruginosa, and H. influenza, and clinically relevant resistance genes. These tests are culture-independent and allow for direct from blood testing where PAXgene tubes stabilize the RNA. The PAXgene Blood RNA Tubes (QIAGEN, MD, USA; Cat. No./ID: 762165) are used for in vitro diagnostic testing (IVD). Sensitivity and specificity align with the requirements of the FDA as it relates to molecular diagnostic tests. Because RNA is used, phenotypic identification is done better than attempts at DNA sequencing. See BMJ Global Health, 5(11) (2020).

Conceptual innovation. Reads that do not align to the genome of interest (human in these assays) are typically discarded. In this invention, the unmapped reads are the focus of the investigation to identify novel PCR targets in patients with bacterial infections.

Deep RNA sequencing, greater than 100 million reads, allows for identifying bacterial RNA in the blood of patients with infections.

Focusing on RNA rather than DNA improves phenotypic correlation with antimicrobial resistance.

Globin and ribosomal RNA are reduced to enhance the identification of the bacterial genes expressed.

Clinical management is guided by these RNA-based PCR tests designed to identify target genes directly affecting treatment decisions.

The RNA-based PCR tests are developed with the goal of rapid dissemination to clinical microbiology laboratories.

Technical innovation. Unmapped reads from deep RNA sequencing are an untapped resource of new information. Typically, 30% of reads are unmapped, so deep RNA sequencing of 100 million reads has 30 million reads for further analysis.

The invention uses analytical algorithms that include mapping reads to genomes created for each pathogen based on standard features across large numbers of strains.

The computational analysis is enhanced with customized algorithms and improved computing power, shortening the time to primer identification.

Workflow is optimized and automated to protect RNA, including PAXgene tubes for phlebotomy.

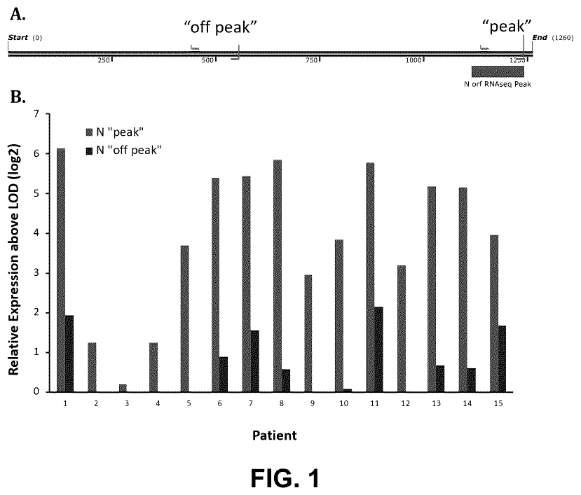

Deep RNA sequencing identifies pathogen RNA and informs PCR primer. Preliminary data was created using RNA sequencing from COVID-19 patients in the ICU. Data from the deep RNA-sequencing assays indicated limited regions of the viral genome were detected in the bloodstream of COVID-19 patients. This information was used to design primers to validate the sequencing results with a different methodology. cDNA generated from patients' RNA was subjected to quantitative, real-time, reverse transcriptase PCR using two sets of primers for the N gene. One primer pair corresponded to the peak of sequencing reads. Another primer pair was selected at a different site of the gene. See FIG. 1A. Using standard SYBR green methodology, amplicons for the peak N sequence were identified in all tested samples. A template corresponding to the off-peak sequence was detected in only nine of the fifteen patients. When present, the abundance of the off-peak sequence was 4 to 16-fold lower than the peak sequence. See FIG. 1A.

This work is important because detecting SARS-COV-2 in the blood was difficult. Yan, Chang, & Wang (2020).

Deep RNA sequencing can identify RNA from pathogens of interest. Deep RNA sequencing data was taken from two patients with bacteremia due to E. coli infection. The unmapped reads were aligned to the E. coli genome. Each patient had reads that aligned to fourteen genes in TABLE 1. Bacterial ribosomal RNA was identified because the depletion kits are designed for human ribosomal RNA. Although previous work looked at ribosomal RNA for pathogen identification, this method is different because the inventors are looking at RNA and not DNA so the inventors can look for actively expressed genes. Like the probe design for SARS-COV-2 above, the inventors identify an exact region of the gene of interest covered by the RNA reads identified by the sequencing data. They can also target multiple genes with PCR based on those with the most reads in sick patients. Interestingly, the patient with more reads died, while the other patient survived. This increase in inventors read counts based on the clinical deterioration could be like a molecular equivalent of time to a positive culture, which is sometimes used clinically. Bläckberg et al. (2022).

Patient 2 died of an ESBL Escherichia coli bacteremia. In this patient, genes CTX-M (twelve counts) and blaCTX-M (twelve counts) were identified. These genes result in an ESBL pathogen, confirming the culture diagnosis.

These data show the ability to isolate RNA from the blood, sequence the RNA, and use computational approaches to identify bacterial sequences and create PCR primers to identify infection and resistance.

TABLE 1
Reads of E coli genes per patient with E coli bacteremia

| Gene | Patient 1 | Patient 2 |
|---|---|---|
| rrlA 23S | 244 | 12645 |
| rrsB 16S | 385 | 12274 |
| rrlC 23S | 232 | 12205 |
| rrsE 16S | 384 | 12179 |
| rrsA 16S | 369 | 12036 |
| rrsC 16S | 446 | 11447 |
| rrlE 23S | 282 | 11044 |
| rrlB 23S | 294 | 10629 |
| rrlD 23S | 248 | 10462 |
| rrlH 23S | 208 | 10335 |
| rrsG 16S | 314 | 9875 |
| rrsD 16S | 308 | 9717 |
| rrlG 23S | 285 | 9340 |
| rrsH 16S | 444 | 7912 |
| (bla)CTX-M | Negative | Positive |

TABLE 2
Test Characteristics of Greatest Importance

| Description | Application in this Invention |
|---|---|
| Rapid: proposed ID test time | A PCR-based test to return a result |
| of <4 hours for clinical | in less than four hours. This test is |
| diagnostics that differentially | done on a sample directly from blood |
| detect and identify species and | by looking at the RNA present from |
| concurrently or sequentially | the bacteria to detect and identify the |
| provide phenotypic resistance | species. Because it is looking at RNA, |
| profile information. | not DNA, a phenotypic resistance |
| | profile is achieved. |
| Culture-independent: the | The pathogen identification results |
| diagnostic should focus on | from information obtained directly |
| direct detection of the target | from the blood. The PCR targets |
| pathogens from primary | pathogen RNA, more abundant than |
| samples | single copies of DNA per organism. |
| | Culture is not needed. |
| Technologies capable of | From samples from patients with |
| detecting drug resistance/ | infections due to resistant organisms, |
| drug-susceptibility, relevant | RNAs corresponding to the resistance |
| to clinical decision making | gene are measured in the PCR. |
| | Resistant genes are selected that |
| | alter antimicrobial therapy for that |
| | pathogen if they are detected. |
| Sensitive and specificity | Sensitivity and specificity are studied |
| should equal or exceed the | and reported according to the Statistical |
| sensitivity and specificity | Guidance on Reporting Results from |
| of FDA-cleared tests for | Studies Evaluating Diagnostic Tests - |
| proposed agents from the | Guidance for Industry and FDA Staff. |
| same sample type. | Culture-based tests are performed in |
| | parallel with the molecular tests being |
| | developed. |

In one aspect, unmapped RNA reads from patients with infections that align with pathogens can inform a better diagnostic test. The invention uses deep sequencing (>100 million reads) to identify the most highly expressed RNAs in the blood of patients with bacteremia or pneumonia. Cultures and antibiotic susceptibility testing are performed as the gold standard. RNA sequences from pathogens in transcription analysis are typically discarded because they would not align with the human genome. The inventors identify these “unmapped reads” in patients' blood and align them to a custom “genome” derived from the pathogens of interest to identify the causative organism. RNA that aligns with resistance genes is also specified. The commonly identified and organism-specific sequences are the template for designing oligonucleotide primers for RT-qPCR tests.

TABLE 3

| | Method | Result | Impact |
|---|---|---|---|
| A1a | Deep RNA sequenc- | PCR is validated to | Common bacteria |
| and | ing identifies RNA | test for bacteremia | causing bacteremia |
| A1b | to target pathogens | caused by S. aureus, | and pneumonia are |
| | in infected patients | E. coli, | identified more |
| | with bacteremia | P. aeruginosa | quickly, allowing |
| | due to S. aureus, | in samples from | faster administra- |
| | E. coli, | patients with and | tion of tailored anti- |
| | P. aeruginosa to | without blood | biotics, improving |
| | create PCR | infections | survival, and enhanc- |
| | | | ing antimicrobial |
| | | | stewardship. |
| A2a | Deep RNA sequenc- | PCR is validated to | |
| and | ing identifies RNA | test for pneumonia | |
| A2b | to target pathogens | caused by S. aureus, | |
| | in infected patients | P. aeruginosa, or | |
| | with pneumonia | H. influenzae in | |
| | due to S. aureus, | samples from | |
| | P. aeruginosa, or | patients with and | |
| | H. influenzae to | without lung | |
| | create PCR | infections | |
| A3a | Deep RNA sequenc- | PCR is validated to | Infections due to |
| and | ing identifies RNA | test for clinically | resistant organisms |
| A3b | to target related to | relevant resistance | are identified quickly. |
| | clinically relevant | genes to S. aureus, | Antibiotics are used |
| | resistance genes to | E. coli, | immediately, reducing |
| | create PCR | P. aeruginosa, | the use of broad- |
| | | or H. influenzae | spectrum antibiotics. |
| | | in samples from | |
| | | patients with and | |
| | | without resistant | |
| | | infections | |

TABLE 4
Microbiological Culture Positivity Rates

| Bacteria | Blood Culture | BAL |
|---|---|---|
| S. aureus | 2.2% | 11% |
| E. coli | 1% | N/A |
| P. aeruginosa | 0.3% | 9.5% |
| H. influenza | N/A | 3.7% |

### Definitions

For convenience, the meaning of some terms and phrases used in the specification, examples, and claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

Acute respiratory distress syndrome (ARDS) has the biomedical art-recognized meaning. ARDS is a type of respiratory failure characterized by the rapid onset of widespread inflammation in the lungs. Symptoms include shortness of breath, rapid breathing, and bluish skin coloration. Causes may include sepsis, pancreatitis, trauma, pneumonia, and aspiration.

Alternative splicing (AS) has the biomedical art-defined meaning. RNA splicing is a molecular function that occurs in all cells directly after RNA transcription but before protein translation, in which introns are removed and exons are joined. Alternative splicing or alternative RNA splicing, or differential splicing, is a regulated process during gene expression that results in a single gene coding for multiple proteins. Exons of a gene can be included within or excluded from the final, processed messenger RNA (mRNA) produced from that gene. The proteins translated from alternatively spliced mRNAs can contain differences in their amino acid sequence and, often, in their biological functions.

COVID-19 has the biomedical art-recognized meaning. The SARS-COV-2 global pandemic has significantly affected global public health. The viral genome is pertinent and relatively small at about 30 kb. Two large overlapping open read frames that encode 16 nonstructural protein as well as four open reading frames that encode structural proteins. The small size of the genome and few number of gene regions makes for a good size for our analysis. Wu et al., Virology Journal, 20(1), 6 (2023). The outbreak of SARS-COV-2 that became the COVID-19 global pandemic has had an enormous impact on global health and economics. Cascella et al., Features, Evaluation, and Treatment of Coronavirus (COVID-19). SARS-COV-2 remains an ongoing threat to human health. El-Sadr, Vasan, & El-Mohandes, N. Engl. J. Med., 388(5), 385-387 (2023).

Ensemble free energy has the physical art-recognized meaning. Ensemble free energy is estimated based on a partition function algorithm also included within the RNAfold program. Lorenz et al., ViennaRNA Package 2.0. Algorithms Mol. Biol., 6, 26 (2011); McCaskill, Biopolymers, 29(6-7), 1105-19 (1990); Zuker & Stiegler, Nucleic Acids Res, 9(1), 133-48 (1981).

Mann Whitney U tests have the statistical art-defined meaning. The Mann-Whitney U test (also called the Mann-Whitney-Wilcoxon (MWW), Wilcoxon rank-sum test, or Wilcoxon-Mann-Whitney test) is a nonparametric test of the null hypothesis that it is equally likely that a randomly selected value from one population is less than or greater than a randomly selected value from a second population. This test can investigate whether two independent samples were selected from populations having the same distribution.

Minimum free energy has the physical art-recognized meaning. Minimum free energy can be estimated using the minimum free energy algorithm that produces an optimal structure. Zuker & Stiegler, Nucleic Acids Res, 9(1), 133-48 (1981).

Motif analysis has the biomedical art-recognized meaning. A DNA sequence motif is a subsequence of DNA sequence that is a short similar recurring pattern of nucleotides, and it has many biological functions A DNA motif refers to a short similar repeated pattern of nucleotides that has biological meaning. See Hashim, Mabrouk, & Al-Atabany, Review of different sequence motif finding algorithms. Avicenna J. Med. Biotechnol., 11(2), 130-148 (April-June 2019). The motif analysis tool called XSTREME can be used to input sequences of any length. XSTREME uses two well established motif discovery programs, MEME and STREME, to identify motifs and uses the SEA algorithm for motif enrichment analysis. MEME-ChIP was used to find and analyze motifs and compare with the RNA database. Sequences were entered to the online version 5.5.1 MEME Suite.

mountainClimber is a cumulative-sum-based approach to identifying alternative transcription start (ATS) and alternative polyadenylation (APA) as change points. Unlike many existing methods, mountainClimber runs on a single sample and identifies multiple ATS or APA sites anywhere in the transcript. Cass & Xiao, Cell Systems, 9(4), 23, 393-400.e6 (October 2019).

Next Generation Sequencing (NGS) has the biomedical art-recognized meaning. NGS technology is typically highly scalable, letting the entire genome be sequenced at once. Usually, this is done by fragmenting the genome into small pieces, randomly sampling for a fragment, and sequencing it using various technologies.

Nucleocapsid gene (N gene) has the biomedical art-recognized meaning of a protein that packages the positive-sense RNA genome of coronaviruses to form ribonucleoprotein structures enclosed within the viral capsid. For example, an N gene can be the gene for the SARS-COV2-Nucleocapsid (N2) gene. See Wu et al., Virology Journal, 20(1), 6 (2023).

Principal Component Analysis (PCA) has the biomedical art-defined meaning. The principal component analysis is a statistical procedure that uses an orthogonal transformation to convert a set of observations of possibly correlated variables (entities, each of which takes on various numerical values) into a set of values of linearly uncorrelated variables called principal components.

Read has the biomedical art-defined meaning of reading sequencing results to determine nucleotide base structure.

Read origin protocol (ROP) has the computer-art meaning of a computational protocol to discover the source of all reads, including those originating from repeat sequences, recombinant B and T cell receptors, and microbial communities. The Read Origin Protocol was developed to determine what the unmapped reads represented. Mangul al., Genome Biology 19, 36 (2018). The recent development of the Read Origin Protocol (ROP) has shown unmapped reads align to bacterial, viral, fungal, and B/T rearrangement genomes.

RNA sequencing (RNA-Seq) has the biomedical art-recognized meaning of a sequencing technique that uses next-generation sequencing (NGS) to reveal the presence and quantity of RNA in a biological sample, representing an aggregated snapshot of the cells' dynamic pool of RNAs, also known as transcriptome. In RNA-Seq, reads may align primarily over certain areas and it is common that there are read sequences that are repeatedly identical. Deschamps-Francoeur, Simoneau, & Scott, Handling multi-mapped reads in RNA-seq., Comput. Struct. Biotechnol. J., 18, 1569-1576 (2020). Several factors determine the extent of repeated sequences, especially with the many processing steps in the RNA-Seq procedure. Fu et al., BMC Genomics, 19(1), 531 (2018).

RNAfold is a computer program from the ViennaRNA Package that is used to predict the minimum free energy of the secondary structure of RNA-Seq read sequences. RNAfold uses a loop-based energy model and dynamic program algorithm to estimate the MFE based on an RNA sequence. Lorenz et al., ViennaRNA Package 2.0. Algorithms Mol Biol, 6, 26 (2011).

Sepsis has the medical art-defined meaning of a life-threatening condition that arises when the body's response to infection injures its tissues and organs. Bone et al., Chest, 101, 1644-1655 (1992); Singer et al., JAMA, 315, 801-810 (February 2016).

STAR aligner is the Spliced Transcripts Alignment to a Reference (STAR), a fast RNA-seq read mapper with support for splice-junction and fusion read detection. Using a Suffix Array index, STAR aligns reads by finding the Maximal Mappable Prefix (MMP) hits between reads (or read pairs) and the genome. Different parts of a read can be mapped to different genomic positions, corresponding to splicing or RNA-fusions. The genome index includes known splice junctions from annotated gene models, allowing for sensitive detection of spliced reads. STAR performs local alignment, automatically soft clipping ends of reads with high mismatches. Dobin et al., STAR: Ultrafast universal RNA-seq aligner. Bioinformatics, 29(1), 15-21 (January 2013).

Treatment for sepsis has the medical art-recognized meaning. Sepsis is treatable, and timely implementation of targeted interventions improves outcomes. The Mayo Clinic informs the public that several medications are used in treating sepsis and septic shock. They include antibiotics. Broad-spectrum antibiotics, which are effective against various bacteria, are usually used first. After learning the results of blood tests, a doctor may switch to a different antibiotic that s targeted to fight the specific bacteria causing the infection. Other medications include low doses of corticosteroids, insulin to help maintain stable blood sugar levels, drugs that modify the immune system responses, and painkillers or sedatives.

Whippet (OMICS_29617) is a program that enables the detection and measurement of alternative RNA splicing events of any complexity with computational requirements compatible with a laptop computer. Whippet applies the idea of lightweight algorithms to event-level splicing measurement by RNAseq. The software can help with the analysis of simple to complex alternative splicing events that function in normal and disease physiology. Alternative splicing events with high entropy are identified using Whippet. Sterne-Weiler et al., Molecular Cell, 72, 187-200.e186 (2018). Whippet can generate an entropy value for each gene's identified alternative splicing and transcription event. These entropy values are created with no groups used in the gene expression analysis. To visualize this data, a principal component analysis (PCA) can be conducted to reduce the dataset's dimensionality and obtain an unsupervised overview of trends in entropy values among the samples. Raw entropy values from all samples can be concatenated into one matrix, and missing values were replaced with column means. Mortality can be overlaid onto the PCA plot to assess the ability of these raw entropy values to predict this outcome in this sample set. This analysis was done in R (version 3.6.3).

Unless otherwise defined, scientific and technical terms used with this application shall have the meanings commonly understood by persons having ordinary skill in the biomedical art. This invention is not limited to the methodology, protocols, reagents, etc., described herein and can vary.

The specification does not concern a process for cloning humans, methods for modifying the germ line genetic identity of humans, uses of human embryos for industrial or commercial purposes, or procedures for modifying the genetic identity of animals likely to cause them suffering with no substantial medical benefit to man or animal and animals resulting from such processes.

Guidance from Materials and Methods

A person of ordinary skill in the biomedical art can use these materials and methods as guidance to predictable results when making and using the invention:

Human subjects. The inventors have timely access to the samples in sufficient quantities. The inventors are enrolling patients in the Intensive Care Units with sepsis and sending their blood for deep RNA sequencing. After Institutional Review Board approval, patients are recruited for this research program from the emergency department and hospital patients when blood cultures are ordered. Through alerts from the electronic health record (EPIC), research assistants are notified of when blood cultures are ordered. Patients have consented before the collection of the blood culture. Samples are drawn in collaboration with the phlebotomy service and the bedside nurse. Blood is collected in two PAXgene tubes, 5 mL of blood, and stored in an −80° C. freezer until RNA is isolated for sequencing. The last six months of data in the hospital were reviewed. Many samples were available. Over the six-month time course, 2,453 patients had blood cultures drawn in the emergency department, and 602 patients had blood cultures drawn in the Intensive Care Units. Blood is also collected from patients who undergo bronchial alveolar lavage (BAL) in the Intensive Care Unit to diagnose pneumonia. Samples are collected before the bronchial alveolar lavage and stored as described. Over the six-month time course, forty-six patients had bronchial alveolar lavage samples obtained in the emergency department and fifty-one patients had bronchial alveolar lavage samples obtained in the Intensive Care Units.

In EXAMPLE 4, research protocols were approved by the Lifespan Institutional Review Board in accord with the Declaration of Helsinki. Participants or their legally authorized representatives provided written informed consent before enrollment.

Biological variables. Both sexes are recruited. Variables such as age (patients are included across the lifespan, weight, and medical co-morbidities are collected and compared across groups. If these variables, or sex, are significantly different (t-test or rank sum), the analysis will adjust these factors via regression.

Variables such as age (patients are included across the lifespan, weight, and medical co-morbidities are collected and compared across groups. If these variables, or sex, are significantly different (t test or rank sum), these factors are adjusted for in the analysis via regression.

Blood sample collection. Blood samples are collected on Day 0 of Intensive Care Unit admission. Clinical data including COVID specific therapies was collected prospectively from the electronic medical record and participants were followed until hospital discharge or death. Ordinal scale can be collected as described by Beigel et al., New England Journal of Medicine (2020); along with sepsis and associated sequential organ failure assessment (SOFA) score, and the diagnosis of ARDS. See Singer et al., The Third International Consensus Definitions for Sepsis and Septic Shock (Sepsis-3). JAMA, 315: 801-810 (2016); Ferguson et al. The Berlin definition of ARDS. Intensive Care Medicine, 38: 1573-1582 (2012).

For the assays described in EXAMPLE 5, blood from patients in the ICU with COVID-19 were collected during 2020 in Paxgene tubes. RNA sequencing was done as described by Fredericks et al., Science Reports, 12(1), 15755 (2022).

RNA extraction and sequencing. Whole blood can be collected in PAXgene tubes (QIAGEN, Germantown, MD, USA) and sent to Genewiz (South Plainfield, NJ, USA) for RNA extraction, ribosomal RNA depletion and sequencing. Sequencing can be done on Illumina HiSeq machines to provide 150 base pair, paired end reads. Libraries were prepared to have three samples per lane. Each lane provided 350 million reads ensuring each sample had >100 million reads.

RNA isolation and sequencing. Blood from patients are collected using the PAXgene tubes (PreAnalytiX, Switzerland). All samples require at least 1400 nanograms of RNA for deep sequencing. With the PAXgene system, one routinely obtains >3000 nanograms. After RNA samples are processed, they are sent out for RNA sequencing. Due to the high concentration of globin and ribosomal RNA in blood samples, these samples are then further processed at the sequencing company to reduce globin RNA and human ribosomal RNA. This optimizes the yield of clinically relevant reads. Each sample are sent out for deep RNA sequencing with a goal of 100 million reads per sample.

RNA sequencing are done on non-CLIA machines because this data is not used in clinical practice. The vendor has Clinical Laboratory Improvement Amendment (CLIA) certified machines to allow for ease of translation in future studies. Not all the blood samples collected are sent for deep RNA sequencing. One of the two PAXgene tubes are kept for the PCR tests.

Sample size calculation. Patients with bacteremia are compared to patients without bacteremia to identify targets for the creation of the PCR. Based on the positive culture rate (TABLE 6), the inventors would collect 2200 blood cultures to obtain fifty that are positive for S. aureus. These rates are for all samples. The inventors are targeting the collection for the Emergency Department and the Intensive Care Units, so the positive rate are higher. 3500 samples are obtained to get a representation of each type of organism. The institution averages 3000 blood cultures in the Emergency Department and the Intensive Care Units every six months. This testing results in at least sixty patients with S. aureus, thirty with E. coli, and ten with P. aeruginosa. All samples with a corresponding positive blood cultures for these three pathogens are sent for deep RNA sequencing. The inventors also send samples for RNA sequencing with a corresponding positive blood culture, including those judged to be contaminants, for about 135, with an additional 115 of samples from patients with negative blood cultures. This process would result in 350 samples sent for RNA sequencing for EXAMPLE 1.

The second PAXgene tube drawn on these patients is used to verify the PCR tests. Patients with pneumonia are compared to patients without pneumonia to identify targets for the creation of the PCR. Based on the positive culture rate (see TABLE 6), the inventors would ideally collect all patients with a bronchial alveolar lavage from the Emergency Department or Intensive Care Unit. Over six months, this process would include about 100 patients and would result in eleven with S. aureus, ten with P. aeruginosa, and four with H. influenza. The inventors collect eighteen months of samples to obtain about 300 blood tubes to sequence for the pneumonia section of the invention. Because two pathogens are being studied, these patients have complementary bronchial alveolar lavage and blood cultures sent simultaneously. Resistance genes are identified using the same samples collected.

Assessment of clinical information. RNA sequencing data are interpreted with clinical data collected from the electronic medical record including endpoints such as mortality, Intensive Care Unit length of stay, hospital length of stay, SOFA score (Shankar-Hari et al. (2016)), ventilator days, renal failure, ARDS (Ferguson et al. (2012)). Culture data are based on the test results in the microbiology lab and are the gold standard. Clinical response to antibiotics are also be tracked to see if the treatment based on microbiology data is correct. Changes in treatment are assessed to ensure culture data is used in treatment and antimicrobial stewardship practices are being followed.

Polymerase chain reaction (PCR) design. Optimized PCR parameters ensure accuracy and reproducibility in qPCR reactions. See Bustin & Huggett (2017); Bustin, Mueller, & Nolan (2020)). The preliminary data show bacterial reads are measurable from patient with bacteremia and pneumonia and that the reads can be aligned to the organism's genome. RNA sequencing data accumulated from patients with bacteremia or pneumonia due to the specified pathogens are used to identify sequences of interest. These sequences are compared to a pan genome of the same organism to confirm the target is generalizable to the pathogen. Wang et al. (2022). The inventors use Beacon Designer (Premier Biosoft) to design several primer/probe combinations for the sequences, the specificity of which are confirmed by BLAST searches. Primers with low specificity, dimer formation, or that create amplicons with complex secondary structures are excluded. Bustin & Huggett (2017). Primer-BLAST (NCBI) are used as an independent, complementary design strategy; primers identified by both approaches are prioritized. PCR reactions are optimized in the laboratory for temperature and primer concentration for the mastermix. The goal is to create a standard set of testing conditions.

Testing the PCR. The PCR tests are validated in two ways. First, cDNA libraries used for RNA sequencing are tested. Next, RNA from the blood of patients, both with and without the infection, are used as templates for cDNA synthesis and then PCR. PCRs are applied to the samples from RNA sequencing and an independent cohort of patients to validate the assays. Several primer combinations are evaluated for each target sequence. Bustin & Huggett (2017). SYBR green methodology are used to prioritize different primer combinations. Hydrolysis (“Taqman”) probes for qPCR, which were already designed with the primers, is then synthesized for the prioritized primer combinations.

Rigor and reproducibility. The preliminary data show isolated RNA from patients and high quality RNA sequencing results. The inventors also focus on isolation methods that are standard and can easily be applied followed so the results can be translated to clinical practice. To enhance robustness during development, it is standard practice for each step of the PCRs (setup, cycling, analysis) to be performed in separate rooms, reducing reactions being contaminated with amplicons from past runs.

Computing resources. Computational biology work is performed on servers on premise. These servers are secured because they contain clinical data. All HIPAA standards are applied. The server operates on 6× VxRail E560F nodes (PowerEdge R640 1U rack mount servers) and has dual Intel Xeon Platinum 8260 (24c) 2.4 Ghz with 1,152 GB RAM, 2×1.6 TB SAS SSD cache, 8×7.68 TB SAS SSD capacity, 4×10 Gb data ports, and 1×1 Gb iDRAC management port. This server includes vSphere Enterprise Plus with 3 Years 24×7 Mission Critical Support per node configured to provide the computational infrastructure. The server consists of 288c (691.2 GHz) CPU, and 6.75 TB RAM. Storage estimates reflect 368.64 TB RAW/222 TB usable memory on a RAID6 configuration with 20% vSAN overhead. This server manages all large data sets from RNA sequencing. Due to the depth of sequencing for RNA splicing analysis (100 million reads vs. 40 million), more data is generated from both sequencing and analysis. In a preliminary project, the inventors generated one terabyte of sequencing data and another terabyte from the alignment to the genome. Because RNA sequencing data is always identifiable, the data from humans are treated as though it is protected health information (PHI), even though none of the typical identifiers (such as name, date of birth, etc.) are associated with the data.

The following pipeline encompasses the typical analysis: differential expression, RNA analysis is done with Whippet (Sterne-Weiler et al. (2018)). After this, the unmapped reads are analyzed for microbial RNA. The inventors curate a reference genome of all identified species of S. aureus, E. coli, P. aeruginosa, and H. influenza. This are done using genomes described in TABLE 4 with the addition of plasmids. Bacterial rearrangements are common across strains. This tool adjusts for rearrangement with a consensus genome to align the un-mapped reads to them. Noureen, Tada, Kawashima, & Arita (2019). This tool allows for visualization and construction of a consensus genome. The conserved and strain specific sequences are kept. Tada, Tanizawa, & Arita (2017). Targets are preferentially chosen from conserved regions. Strain specific targets are used if clinically relevant. Specific resistance genes are also be searched for in the unmapped reads using the STAR aligner.

Cloud based computing. Due to the depth of sequencing RNA splicing analysis (100 million reads vs. forty million), more data is generated from both sequencing and analysis (a small study generated one terabyte of sequencing data and another terabyte from the alignment to the genome). With such a large amount of data predicted, the ability to expand and contract the storage space and computing power in the cloud is the ideal choice. This server stores and analyzes data from both mouse and human samples. Because RNA sequencing data is always identifiable, the data from humans are treated as though it is protected health information (PHI), even though none of the typical identifiers, such as name, date of birth, etc., are associated with the data. The cloud server is only accessible through a hospital virtual desktop and data are saved only to the Azure server or a hospital computer. Data are encrypted while stored, and when in transit to or from the hospital. Any link to typical identifiers are kept separate from the sequencing data. The cloud-based server allows for large data analysis with computing and storage needs changing on a per-use basis. The Azure server is Linux based and uses programming in R and Python. The following pipeline encompasses the typical analysis: differential expression, RNA analysis is done with Whippet. This also includes an entropy measure, and genes of interest undergo gene ontology term analysis. Genes with alternative transcription start and end sites identified through Whippet are correlated with findings from the mountainClimber analysis.

Computational analysis and statistics. RNA sequencing data was first checked for quality using FASTQC. RNA sequencing data collected from the GTEx consortium and analyzed with the Whippet software for differential gene processing. Alternative transcription events are those events identified by Whippet as ‘tandem transcription start site,’ ‘tandem alternative polyadenylation site,’ ‘alternative first exon,’ and ‘alternative last exon.’ Alternative RNA splicing events are those events labeled ‘core exon,’ ‘alternative acceptor splice site,’ ‘alternative donor splice site,’ and ‘retained intron.’ Alternative mRNA processing events were determined by a log 2 fold change of greater than 1.5+/−0.2. Statistical significance was calculated by the chi-square p-value of a contingency table based on 1000 simulations of the probability of each result.

Computational biology and statistical analysis. All computational analysis can be done blinded to the clinical data. The data can be assessed for quality control using FastQC. See Andrews, A quality control tool for high throughput sequence data. FastQC (2014). RNA sequencing data can be aligned to the human genome using the STAR aligner. Dobin et al., Bioinformatics (Oxford, England), 29, 15-21 (2013). Reads that aligned to the human genome can be separated and called ‘mapped’ reads. Reads that do not align to the human genome, which are typically discarded during standard RNA sequencing analysis, were identified as ‘unmapped’ reads. The unmapped reads then align to the relevant comparator and counted per sample using Magic-BLAST. See Boratyn et al., BMC Bioinformatics, 20, 405 (2019). The unmapped reads were further analyzed with Kraken2. See Wood, Lu, & Langmead, Genome Biology, 20, 257 (2019). The analysis used the PlusPFP index to identify other bacterial, fungal, archaeal, and viral pathogens. See Kraken2/Bracken Refseq indexes maintained by BenLangmead, which uses Kutay B. Sezginel's modified version of the minimal GitHub pages theme.

Reads that align to the human genome, the mapped reads, also can undergo analysis for gene expression, alternative RNA splicing, and alternative transcription start/end by Whippet. See Sterne-Weiler et al., Molecular Cell, 72, 187-200.e186 (2018). When comparisons are made between groups (died vs. survived) differential gene expression can be set with thresholds of both p<0.05 and +/−1.5 log 2 fold change. Alternative splicing was defined as core exon, alternative acceptor splice site, alternative donor splice site, retained intron, alternative first exon and alternative last exon. Alternative transcription start/end events can be defined as tandem transcription start site and tandem alternative polyadenylation site. Alternative RNA splicing and alternative transcription start/end events can be compared between groups. See Sterne-Weiler et al., Molecular Cell, 72, 187-200.e186 (2018). Significance was set at great than 2 log 2 fold change as described by Fredericks et al., Intensive Care Medicine (2020). Genes identified from the analysis of mapped reads can be evaluated by GO enrichment analysis (PANTHER Overrepresentation released 20200728). See Mi et al. Nature Protocols, 8, 1551-1566 (2013).

Kraken2. These tools are compatible with both Kraken1 and Kraken2. Both tools help users in analyzing and visualizing Kraken results. Bracken lets users estimate relative abundances within a specific sample from Kraken2 classification results. Bracken uses a Bayesian model to estimate abundance at any standard taxonomy level, including species/genus-level abundance. Pavian has also been developed as a comprehensive visualization program that can compare Kraken2 classifications across multiple samples. KrakenTools is a suite of scripts to help analyze Kraken results. For more information, a person having ordinary skill in the biomedical art can refer to Wood, Lu, & Langmead, Improved metagenomic analysis with Kraken2, Genome Biology (Nov. 28, 2019).

In EXAMPLE 5, the inventors present an analysis of the stability of targets to diagnose COVID-19. RNAfold from the ViennaRNA Package was used to predict the minimum free energy of the secondary structure of RNA-Seq read sequences. RNAfold was also used to calculate the minimum free energy value of the structures and ensemble free energy values to compare stability between different read sequences. Energy parameters for calculations were set at 37° C. Different statistical tools were used to assess the stability or instability of a secondary structure in addition to RNAfold.

Sequences that were outliers in length were disregarded for analysis. Only sequences that were less than 175 nucleotides long were analyzed. Energy parameters for calculations were set at 37° C. Statistical analysis was computed using R Studio. A Welch ANOVA was first performed to compare the minimum free energy (MFE) values and ensemble free energy (EFE) values for reads that were located within known gene regions.

Allocation to a certain gene region was determined by which gene region the middle of the read sequence was located. A Games-Howell post hoc test was performed for pairwise comparison of free energy values between genes. A similar statistical analysis was conducted on the nucleocapsid (N) gene.

A Welch T-test was performed to compare the minimum free energy and ensemble free energy of the sequences with and without the motif. Sequences inputted into MEME-ChIP are ideally 500 letters in length while the longest read sequences used in our analysis was 151 nucleotides long. MEME-ChIP was used to find only the first ten motifs. Analysis of the effect of a destabilizing motif on quantity of duplicated reads was conducted using a negative binomial regression.

A Welch ANOVA was first performed to compare the minimum free energy values and the ensemble free values for reads at the beginning, middle, or end of the N gene. The N gene was divided into three equally long regions, and allocation to the region of the N gene was determined by the middle of the read sequence. A Games-Howell post hoc test was performed for pairwise comparison of free energy values between regions of the N gene. Because the early gene region for the N gene had zero variance, a Welch t-test was conducted to compare individual within gene regions. A Chi-Squared goodness of fit test was performed to assess the distribution of reads among the genes. This established if certain genes had more or fewer reads relative to other genes.

Gene ontology (GO) was assessed using The Gene Ontology Resource Knowledgebase. Ashburner et al., Nature Genetics, 25, 25-29 (2000); The Gene Ontology Resource. Nucleic Acids Research, 47, D330-d338 (2019). Genes from the analyses were entered, and outputs were displayed. Outputs from gene ontology do not correlate with actual increase or decrease in a gene's expression but are related to expected based upon the set of genes entered.

Pipeline. The following pipeline encompasses the typical analysis: differential expression, RNA analysis is done with Whippet (Sterne-Weiler et al. (2018)). After this, the unmapped reads are analyzed for microbial RNA. We curate a reference genome of all identified species of S. aureus, E. coli, P. aeruginosa, and H. influenza. Bacterial rearrangements are common across strains. This tool adjusts for rearrangement because we make a consensus genome to align the unmapped reads to them. Noureen, Tada, Kawashima, & Arita (2019). This tool allows for the visualization and construction of a consensus genome. The conserved and strain-specific sequences are kept. Tada, Tanizawa, & Arita (2017). Targets are preferentially chosen from conserved regions. Strain-specific targets are used if clinically relevant. Specific resistance genes are also searched for in the unmapped reads using the STAR aligner.

The following EXAMPLES are provided to illustrate the invention and should not be considered to limit its scope in any way.

### Example 1

Design a Direct from Blood, without Culture, Reverse Transcriptase Polymerase Chain Reaction (RT-qPCR) Test for Bacteria Causing Bacteremia, Specifically S. aureus, E. coli, P. aeruginosa, Based on the RNA Identified in Patients with Bacteremia Caused by these Organisms (A1a).

Rationale. Blood cultures are the current gold standard for pathogen diagnostics but take days. Blood cultures have a known contaminant rate, which can adversely affect treatment and disease progression, as shown in the COVID pandemic. Yu et al. (2020).

RNA sequencing is an emerging technology that can enhance the diagnostic capabilities. Unmapped reads, i.e., reads that do not align to the human genome, are typically discarded in RNA sequencing data from humans. When the depth of the RNA sequencing is enough, these unmapped reads can provide useful clinical information. The unmapped reads found in the blood of patients with bacteremia are used to inform the development of a diagnostic PCR.

The gene expression of the bacteria discriminates between infection and simply colonization. D'Mello et al. (2020). Targeting RNA is more specific than DNA by eliminating the signals of free DNA from dead bacteria or pathogen DNA released from immune cells combating the infection. Opota, Jaton, & Greub (2015).

TABLE 5
Creation of Bacterial Genomes

| Bacteria | Genomes | Plasmids |
|---|---|---|
| S. aureus | https://www.ncbi.nlm.nih.gov/genome/154 | 958 |
| E. coli | https://www.ncbi.nlm.nih.gov/genome/167 | 5895 |
| P. aeruginosa | https://www.ncbi.nlm.nih.gov/genome/187 | 155 |
| H. influenza | https://www.ncbi.nlm.nih.gov/genome/165 | 1 |

Assay 1. Assess the RNA sequencing data from patients with blood infections due to S. aureus, E. coli, and P. aeruginosa. Unmapped reads or reads that do not align to the organism of interest, are typically discarded. These reads are used to identify bacterial RNA in the blood. This was initially done using Kraken2. Wood, Lu, & Langmead (2019). For more granularity, the inventors assembled custom genomes to which the unmapped reads are aligned using STAR RNA-sequencing aligner. Dobin et al. (2013). These genomes are based on the common genome in TABLE 4 but also include sequences from other chromosomes and the plasmids attributed to those bacteria, creating a pan-genome. Eizenga et al. (2020). Samples from patients with S. aureus are used to identify the significant reads that align to this bacterium and repeated for other pathogens of interest. This gives a total read count for each bacterium and the parts of the genome with the most abundant reads. From these abundant reads, PCR primers test for the pathogens based on large reads of common areas across many patients.

Assay 2. Create RT-qPCR primers to identify S. aureus, E. coli, and P. aeruginosa causing bacteremia. Using the targets of interest from the deep RNA sequencing data, PCR primers cover these parts of the bacterial genome identified. Multiple primers for multiple targets can identify one pathogen, however this are done through multiplexing with the NeuMoDx instrument from the industry partner. The target of these primers are the RNA in the blood, a reverse transcriptase reaction are used to create the cDNA for the PCR.

Expected results. The preliminary data show that patients with bacteremia have bacterial RNA in their blood that correlates with the causative organism (TABLE 1) There should be a set of highly expressed genes from each of the bacteria during infection that can be the basis for identification. The inventors expect genes like the coagulase gene to be detected in patients with S. aureus bacteremia. Cheng et al. (2010). The inventors prioritize PCR targets unique to the bacteria being tested and distinct from other bacteria. More findings include observations that gene expression of the bacteria can also determine colonization versus infection based on expression pattern and abundance. D'Mello et al. (2020). The number of reads, i.e., transcript abundance, may correlate with patient condition or patient outcomes. Abundant clinical data is associated with patients from with the samples are derived. Read frequency or abundance on RT-qPCR are evaluated for these correlations.

Potential alternatives. The bacteria identified in the sequencing may not be correlated to microbiology culture. This could be due to blood cultures being negative in 50% of blood stream infections, due to low numbers of bacteria in the blood or the impact of antibiotics before the sample is obtained. Opota, Jaton, & Greub (2015). Blood culture could identify the wrong pathogen while another pathogen could cause infection, i.e., a contaminant. the approach includes aligning unmapped reads using Kraken2 to identify background levels of sequences from unrelated bacteria that could be commensals or contaminants. A single gene may not uniquely identify an organism, reducing specificity of the test. In that situation, the inventors test gene combinations as described above. Alternatively, unique alleles/SNPs are used to define a specific pathogen. Established techniques are used to measure SNPs in the RT-qPCR format.

### Example 1A

Validate these RT-PCR Tests in Samples from Patients with and without Bacteremia (A1b).

Rationale. RT-qPCR allow for the identification of pathogens, directly from blood, without culture, in less than four hours. RT-qPCR also allow for faster, pathogen-directed antibiotic selection. Blood culture collection is recommended before antibiotic administration to enhance the diagnostic sensitivity of the blood culture. See Evans et al. (2021). With this diagnostic test proposed here, antibiotics are not expected to influence the RNA present at the blood draw.

Assay 1. Test PCR primers on samples used for RNA sequencing. The cDNA libraries created for RNA sequencing are accessed as the initial test of the PCR primers. Because the RNA sequencing data determined these RNA segments were present, this are the first step in assessment of the utility of these novel PCR tests for the bacteria. The cDNA from all samples with positive cultures for each of the bacteria are used for this assay. Each cDNA sample are tested using the PCR primers for all pathogens. As a negative control for specificity, the inventors also cDNA from the blood of patients and normal controls that have no infections.

Assay 2. Validate PCR primers on samples that mimic collection for clinical use. To obtain sensitivity and specificity in line with FDA requirements, samples from patients with and without confirmed blood infections due to the pathogens of interest are identified from banked clinical specimens, including PAXgene tubes. The PAXgene tubes are being collected at the time of blood culture collection and stored. In the experience, PAXgene tubes stabilize high quality RNA. To enhance robustness of the testing, these tubes are blinded to the team performing the PCR assays. The RNA are extracted and globin and rRNA are reduced using commercially available kits. cDNA libraries are made with reverse transcriptase and then PCR are done with the primers. PAXgene tubes are used but the RT-qPCR are done immediately to ensure the result returns in less than four hours.

Expected results. A panel of PCR primers are developed and optimized on a machine that can be easily translated to a clinical microbiology laboratory. The tests identify S. aureus, E. coli, and P. aeruginosa directly from the blood through extraction of RNA, reduction of globin and rRNA, and creation of cDNA for the PCR. These tests have sensitivity and specificity in line with requirements of the FDA. These direct from blood PCR are initially done for S. aureus, E. coli, and P. aeruginosa. Through collecting samples from patients in PAXgene tubes with other infections, the PCR panel can be expanded as new targets from more pathogens are identified. PCR is rapid and could monitor treatment impact, a practice not currently done as culture takes days to return. If successful treatment is detected, antibiotic course could be shortened and enhance antimicrobial stewardship.

Potential alternatives. Blood interferes with PCR when identifying DNA, but not RNA. Sidstedt et al. (2018). Deep RNA sequencing may find a gene that identifies infection, but PCR conditions cannot be optimized to replicate the finding. This problem can be solved when RNA sequencing costs and time are reduced. RNA sequencing should take less than four hours at a depth of 100 million reads or more.

### Example 2

Design a Direct from Blood, without Culture, Reverse Transcriptase Polymerase Chain Reaction (RT-PCR) Test for Bacteria Causing Pneumonia, Specifically S. aureus, P. aeruginosa, H. influenzae, Based on the RNA Identified in Patients with Pneumonia Caused by these Organisms (A2a).

Rationale. The diagnosis of hospital-acquired pneumonia is complex. Modi & Kovacs (2020). Bronchial alveolar lavage (BAL) is the gold standard, like blood culture for bacteremia. Because bronchial alveolar lavage requires an invasive intervention (bronchoscopy) that can worsen the clinical picture, screening tools are used to decide when to perform them, hence the yield is higher than blood culture. A direct from blood test that provides the same diagnosis obviates the need for the invasive bronchoscopy intervention. The assays described below parallel those in EXAMPLE 1 with an independent cohort of patients diagnosed with pneumonia and who undergo BAL.

Assay 1. Assess the RNA sequencing data from patients with pneumonia due to S. aureus, P. aeruginosa, and H. influenzae. As described above for the blood infections, unmapped reads are aligned to genomes of interest (TABLE 4) to identify genes with increased expression in patients with infection diagnosed by BAL. The blood are collected in PAXgene tubes at the time of BAL. S. aureus and P. aeruginosa genes that are identified are compared to the genes identified for bacteremia. From these reads, PCR primers are developed.

Assay 2. Create RT-PCR primers to identify S. aureus, P. aeruginosa, and H. influenzae causing pneumonia. Using the reads generated from Assay 1, PCR primers are developed to identify pathogens causing hospital acquired pneumonia and applied to the sequenced samples and an independent cohort.

Expected results. The preliminary data show that patients in the ICU have bacterial RNA in the blood. There are a set of highly expressed genes from the bacteria during infection that can be the basis for identification. One outcome is that these genes differ from the genes expressed during bacteremia because some suggested that gene expression changes from the bacteria based on site of infection/colonization. The inventors have RNA sequencing data from patients with bacteremia and pneumonia due to similar pathogens and can see if different genes are expressed at higher rates. Primers can be developed for each pathogen based on site of infection to guide diagnosis. An alternative outcome is that the same target sequences are found in bacteremia and pneumonia. This would simplify product development on the NeuMoDx and require the test be integrated into other clinical diagnostics such as X-rays.

Potential alternatives. The technical approach is like EXAMPLE 1, which the inventors have shown is possible. See TABLE 1. Because the infection is in the lung, there may be no bacterial RNA identified in the blood of these patients, but other studies dispute this possibility. D'Mello et al. (2020). Bacterial DNA was detected in the blood of pneumonia patients. Langelier et al. (2020). The lung has a large surface area for gas exchange that would help with transfer of stable RNA or RNA in micro vesicles from the infection into the bloodstream. Blenkiron et al. (2016). Bacteremia complicates pneumonia in 6-17% of cases, depending on severity. Zhang, Yang, & Makam (2019). A subset of the pneumonia patients are expected to have target sequences shared with the patients studied in EXAMPLE 1. If the sequencing analysis cannot distinguish between the subgroups of pneumonia patients with and without bacteremia, clinical data are used to guide management.

### Example 2A

Validate these RT-qPCR Tests in Samples from Patients with and without Pneumonia (A2b).

Rationale. The PCR targets identified by sequencing can be used clinically. Hospital acquired pneumonia typically rapidly deteriorates a patient. RT-qPCR allows for the identification of pathogens, directly from blood, without culture, in less than four hours, and faster selection of pathogen-directed antibiotics. The goal is to eliminate invasive bronchoscopies, which can delay antibiotic administration and increase risks to the patient.

Assay 1. Test PCR primers on samples used for RNA sequencing. RNA sequencing cDNA libraries again are the initial test of the PCR primers. The cDNA from all samples with positive bronchial alveolar lavage cultures for each of the bacteria are used for this assay. Each cDNA sample are tested using the PCR primers for all pathogens. The inventors also use cDNA from patients that had no hospital-acquired pneumonia.

Assay 2. Test PCR primers on samples that mimic collection for clinical use. This test is done obtain sensitivity and specificity in line with FDA requirements. PAXgene tubes from patients with and without confirmed hospital acquired pneumonia due to the pathogens of interest are identified. The PAXgene tubes are collected when bronchial alveolar lavage is collected. The patients' infection status are blinded to the researchers performing the PCRs. The stabilized RNA are extracted from the PAXgene tubes and globin and human rRNA are depleted using a commercial kit from New England Biolabs. cDNA are made with reverse transcriptase and then PCR are done with the primers. PAXgene tubes are used. RT-PCR are done immediately to ensure result in less than four hours.

Expected results. Specific RT-qPCR assays validate the sequencing and diagnose hospital-acquired pneumonia due to S. aureus, P. aeruginosa, and H. influenzae from a direct from blood sample in fewer than four hours. Target abundance vary among patients (see, e.g., TABLE 1), which are correlated with severity of the pneumonia. Efforts are directed to finding primers that diagnose pneumonia and that are distinct from those for bacteremia despite being due to the same pathogen.

### Example 3

Using the RNA from patients with infections, design an RT-qPCR for the most common resistance genes expressed that would influence treatment for S. aureus, E. coli, P. aeruginosa, and H. influenzae (A3a).

Rationale. Delays in antibiotics worsen outcomes for all patients, including those with resistant organisms. Bonine et al. (2019). Overtreatment of organisms that do not carry resistance determinants also worsens outcomes. Rhee et al. (2020). The goal of this EXAMPLE is to harness data from RNA sequencing to inform PCR-based diagnostics of antimicrobial resistance that are clinically relevant. Reads from the sequencing studies described above are aligned to a “genome” of resistance genes of interest, then novel PCR primers test against clinical specimens. These are “phenotypic” measurements of antibiotic resistance because gene expression and resistance phenotypes are closely linked. Suzuki, Horinouchi, & Furusawa (2014).

Assay 1. Assess the RNA sequencing data from patients with infections due to S. aureus, E. coli, and P. aeruginosa, and H. influenza for resistance genes. A “genome” are made using clinically relevant resistance genes. For S. aureus, the inventors include mecA (methicillin resistance) (Chambers & Deleo (2009); Guo et al. (2020)), qacA, norA, smr (efflux transporters of quinolones and tetracyclines) Guo et al. (2020), beta-lactamase (hydrolyses cefazolin) (Guo et al. (2020)), and VRSA (vanA, vanB, vanC, vanX, vanY, vanA). E. coli targets include multiple beta-lactamases: basic beta-lactamases cleaving ampicillin, ESBL genes (TEM-1, TEM-2, and SHV-1) CTX-M (see TABLE 1), ampC, carbapenemases: KPC (class A), metallo (class B: IMP, VIM, NDM-1), OXA (class D) (Bajaj, Singh, & Virdi (2016), GyrA and ParC (fluoroquinolone resistance) (Tchesnokova et al. (2019), acrB (Karczmarczyk et al., 2011), ompF, Efflux pumps PabetaN, and qnr (Salah et al. (2019)). For P. aeruginosa ampC, oprM, mex Y (efflux transporters for quinolones and aminoglycosides) (Islam et al. (2009)), bla, gyrA, gyrB, parC (for quinolones) (Yang et al. (2015)), and aac(6′)-lb,aphA1, and aadB (aminoglycosides) (Teixeira et al. (2016)). For H. influenzae TEM-1 and ROB-1 (Gutmann, Williamson, Collatz, & Acar (1988); Tristram, Jacobs, & Appelbaum (2007)). (52, 53) Using these genes, PCR primers are identified based on RNA data from patients with these infections. This again are a primer for RT-PCR as the target are RNA. Using RNA as the target yields better results rather than DNA. This tool adapted for use with RNA data could enhance the phenotypic correlation using this data set. Bortolaia et al. (2020).

Assay 2. Create RT-PCR primers to identify resistance genes. Using the targets of interest from the deep RNA sequencing data, PCR primers cover resistance genes. Multiple primers may identify resistance genes for one pathogen. This are done through multiplexing that is possible with the machine from the industry partner. The target of these primers are the RNA in the blood, a reverse transcriptase are used to create the cDNA for which the primers interact. Targeting RNA has a better phenotypic correlation than targeting DNA from the pathogen because RNA signifies that the gene is being actively expressed.

TABLE 6
Antibiotic resistance markers and therapeutic considerations.

| | | Antibiotic if | Antibiotic if | |
|---|---|---|---|---|
| Bacteria | Resistance Gene | present | absent | Ref. |
| S. aureus | mecA | vancomycin | nafcillin | Chambers (1997) |
| | blaZ | nafcillin | cefazolin | Dingle et al. (2022) |
| | VRSA (vanA) | daptomycin | vancomycin | |
| E. coli | ESBL | carbapenem or | ampicillin or | Paterson et al. (2001); |
| | | ceftolozane-tazo | 3GenCeph | Rupp & Fey (2003) |
| | Class A | ceftazidime-avi | ampicillin or | Rivera-Izquierdo et al. |
| | carbapenemase; | or aztreonam or | 3GenCeph | (2021) |
| | Class B | tigecycline | | |
| | carbapenemase; | | | |
| | Class D | | | |
| | carbapenemase | | | |
| | gyrA, parC | ampicillin or | fluoroquinolone | Tchesnokova et al. |
| | | cephalosporin | | (2019) |
| P. | ampC | carbapenem | cefepime/ | Jacoby (2009) |
| aeruginosa | | | ceftazidime | |
| | oprM, mexY | non- | fluoroquinolone | Yang et al. (2015) |
| | | fluoroquinolone | | |
| | gyrA/parC point | non- | fluoroquinolone | Yang et al. (2015) |
| | mutation | fluoroquinolone | | |
| H. | tem-1, rob-1 | amoxicillin-clav | amoxicillin | Gutmann, Williamson, |
| influenza | | | | Collatz, & Acar (1988); |
| | | | | Tristram, Jacobs, & |
| | | | | Appelbaum (2007) |

(tazo, tazobactam; avi, avibactam; 3GenCeph, 3^{rd} generation cephalosporin; clav, clavulanic acid.)

Potential alternatives. In one detailed study of transcription and protein abundance in E. coli, there was a lack of correlation between RNA and protein levels. Taniguchi et al. (2010). Though the overall abundances were not correlated, enzyme transcription and translation were closely correlated. Taniguchi et al. (2010). Some resistance phenotypes, such as fluoroquinolone resistance due to gyrA, gyrB, and parC, are mediated by SNPs. In this situation, the PCR primers are adapted for SNP detection such as the TaqMan assay. Easterday, Van Ert, Zanecki, & Keim (2005). For some resistance mechanisms, such as beta-lactamases, there are too many individual genes to test. In this situation, the inventors use k-mer analysis to identify primers capable of detecting entire classes of beta-lactamases. Marini et al. (2022). Ultimately, there are concerns for whether RNA-based detection of resistance is sufficiently comprehensive to be used in clinical practice. Regulatory RNA may play a role in resistance but not be detected by the sequencing approach. Dersch, Khan, Mühlen, & Görke (2017). In this situation, the inventors evaluate more patient specimens and alter sequencing protocols to detect unconventional RNAs.

### Example 3A

Validate these PCR Tests for Resistance Genes in Samples from Patients with and without Infections (A3b).

Assay 1. Test PCR primers on samples used for RNA sequencing. cDNA libraries used for RNA sequencing are the initial test of the PCR primers. See FIG. 1. The cDNA from all samples with positive cultures with resistance are the positives. Each cDNA sample are tested using the PCR primers for all resistance genes to assess primer specificity.

Assay 2. Test PCR primers on samples that mimic collection for clinical use. These tests are done obtain sensitivity and specificity in line with FDA requirements. PAXgene tubes from patients with and without confirmed infections with resistance are used as negative controls. The assays include a positive control gene like actin to confirm PCR reaction in each specimen.

Expected results. Sets of PCR primers identified in this EXAMPLE detect resistance in these validation studies. The most straightforward tests are for the presence or absence of RNA encoding a resistance mechanism, such as mecA in MRSA. Although a molecular test is used in the clinical microbiology lab to diagnose MRSA, this test requires a positive blood culture bottle. The goal is to confirm an RNA-based blood test that alters treatment described in TABLE 5. That returns results in less than four hours without having to culture the patient's blood.

Potential alternatives. The principal concerns are for the level of target sequence found in blood, i.e., sensitivity, and the ability to identify primers that amplify the expected sequence, i.e., specificity. Strategies for improving sensitivity include using more cDNA in the PCR reaction and conducting a nested PCR. The inventors have not encountered evidence for inhibition of PCR reactions, which is due to the additional processing involved with using RNA as a PCR template. The inventors also continue to use a positive reference gene, such as actin, to test for PCR inhibitors. There may be a large number of potential sequences that could convey a phenotype, such as the large family of beta-lactamases. k-mer analysis are used to identify sequences that represent the family, and primers are designed against that analysis. Modified PCR reactions, such as TaqMAMA, are used when mutations of pre-existing genes convey a resistant phenotype, as SNPs in gyrA and parC that are responsible for fluoroquinolone resistance. Another possibility is that important resistance mechanisms are infrequently encountered in the patient population, such as carbapenemase production. To create a more comprehensive test under those circumstances, the inventors can evaluate appropriate resistant strains in vitro, such as from the CDC & FDA Antibiotic Resistance Isolate Bank that is available to researchers. Another theoretical concern is that the test finds target sequences in patients without infections or normal controls. RT-qPCR holds a distinct advantage over endpoint PCR, so the inventors can establish a threshold cutoff for test positivity using relative abundance measurements of targets by the ΔCt calculation: Ct of assay−Ct of actin gene.

### Example 4

### Improved PCR Primers for SARS-COV-2 Viremia Informed by RNA Sequencing.

COVID-19 is diagnosed using nucleic acid and antigen tests, of which the reverse transcription polymerase chain reaction (RT-PCR) is considered the gold standard. Peeling, Heymann, Teo, & Garcia, Lancet, 399(10326), 757-768 (2022). Although nasopharyngeal swabs are typically used for diagnostic testing, recent studies have shown SARS-COV-2 viremia, or RNAemia. is correlated with disease severity and patient mortality. Fajnzylber, et al., Nature Commun., 11(1), 5493 (2020); Heinrich, F., et al., Open Forum Infect. Dis., 8(11), ofab509 (2021); Jacobs et al., Clin. Infect. Dis., 74(9), 1525-1533 (2022); and Rodriguez-Serrano et al., Science Reports, 11(1), 13134 (2021). Many patients in these studies did not show a detectable viremia, which is in part a reflection of disease severity. Improved sensitivity of a viral load measurement, however, could provide more information about prognosis.

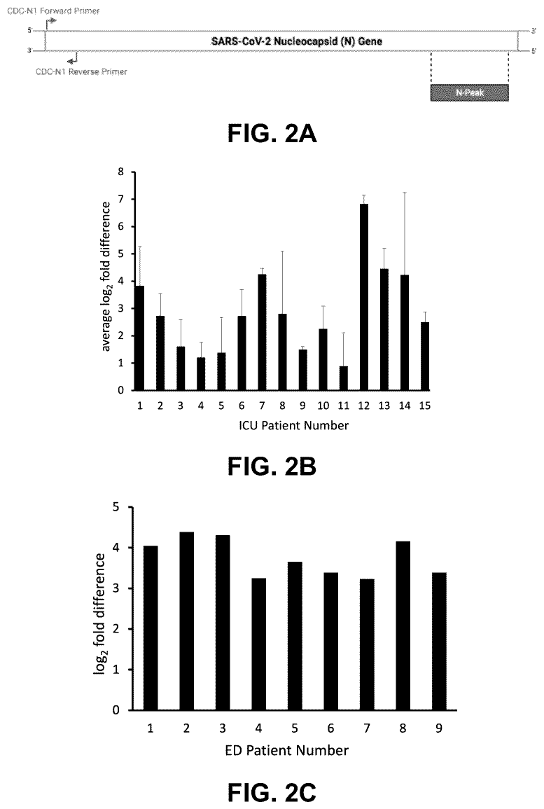

SARS-COV-2 measurements typically use existing assays based on primers designed by the CDC, which were selected for specificity using in silico analyses. Lu et al., Emerg. Infect Dis., 26(8), 1654-65(2020). When these primers were created, there was little information available to inform their design beyond the SARS-CoV-2 sequence. Viral RNA sequences are unevenly represented in the bloodstream of patients with severe COVID-19. Lu et al., Emerg Infect Dis., 26(8), 1654-65(2020). Deep RNA sequencing showed two peaks were overrepresented in the alignments to the SARS-COV-2 genome, suggesting that RT-PCR primers targeting those sites could detect viremia better. In this EXAMPLE, the inventors designed primers to measure the peak of the nucleocapsid (N) gene to be comparable to the widely used CDC-N1 primers (FIG. 2A) with similar GC percentages and amplicon length. BLAST analysis showed similar results as the CDC primers and no significant cross reactivity to other sequences, but they had different locations on the N-gene.

cDNA generated from RNA derived from the original sequenced cohort was first used to compare primers. See Fredericks, Sci. Rep., 12(1), 15755 (2022). Quantitative (q) RT-PCR reactions were performed. Ct values were compared using CDC-N1 primers as the reference. The N-peak primers were about 2-fold to 100-fold more sensitive than the CDC-N1 primers at detecting the N-gene (FIG. 2B).

Because of the inherent variability seen between patients and to validate the findings, viremia was tested in a second cohort of patients. Using a similar approach, the inventors found that N-peak primers were about 10-fold more sensitive than the CDC-N1 primers (FIG. 2C).

Enhanced sensitivity of RNAemia could improve the ROC curve and inform the lower range of viremia measurements. More information could improve assessments of prognosis, especially by those who may progress to develop more severe disease. The results of this EXAMPLE show the value of informing qRT-PCR primer design with RNA sequencing data since certain sequences or genes may be unexpectedly overrepresented during infection in vivo. Enhanced sensitivity could lead to diagnostics using direct-from-blood molecular testing.

RNA was isolated as described by Fredericks, Sci. Rep., 12(1), 15755 (2022). One hundred ng of total blood RNA, depleted of globin and ribosomal RNA, were used for cDNA synthesis with the SuperScript IV First-Strand Synthesis System (Invitrogen, USA) following the manufacturer's instructions in a final volume of 20 μL, and the cDNA was stored at −20° C. until use. Real-time qPCR was performed using iTaq Universal SYBR Green Supermix (Bio-Rad, USA) following the manufacturer's instructions. The final volume of each qPCR reaction was 10 UL, including 1 μL of cDNA, and the final concentration of the primers in each reaction was 400 nM. All qPCR reactions were centrifuged at 455 RCF for one minute before thermal cycling. The qPCR was performed using a CFX Connect or CFX96 instrument (Bio-Rad, USA) controlled by the CFX Maestro Software (Bio-Rad, USA) with the following thermal cycling protocol: 95° C. for thirty seconds and forty cycles of steps: (1) 95° C. for five seconds; (2) 60° C. for thirty seconds.

TABLE 7
Primer sequences used in this EXAMPLE

| | | Forward Primer | Reverse Primer |
|---|---|---|---|
| Name | Target Gene | Sequence 5′-3′ | Sequence 5′-3′ |
| N-Peak | SARS-CoV-2 | ATGAAACTCAAGCCTTA | ATCCAAATCTGCAGCAG |
| | Nucleocapsid | CCGCA | GAAG |
| | gene | | |
| CDC- | SARS-CoV-2 | GACCCCAAAATCAGCGA | TCTGGTTACTGCCAGTT |
| N1 | Nucleocapsid | AAT | GAATCTG |
| | gene | | |
| ACTB | Human beta-actin | GCACCACACCTTCTACA | ATAGCACAGCCTGGATA |
| | gene | ATGAG | GCAAC |

Threshold cycle counts (Ct) were measured where beta-actin was the reference gene and the CDC-N1 primers were used as the calibrator.

TABLE 8
Calculations used in this EXAMPLE

| Value | Calculation |
|---|---|
| Delta Ct (ΔCt) | ΔCt = Ct (N-Peak or CDC-N1) − Ct (ACTB) |
| Delta Delta Ct (ΔΔCt) | ΔΔCt = ΔCt (N-Peak) − ΔCt (CDC-N1) |
| Relative Fold Difference | relative fold difference = 2^{-(ΔΔCt)} |
| Log₂ Relative Fold | log₂ relative fold difference = log₂(2^{-(ΔΔCt)}) |
| Difference | |

### Example 5

Stability and Motif Analysis of RNA-Seq Reads from COVID-19 Patients.

RNA sequencing has been increasingly incorporated in clinical diagnoses and management. See Ketkar, Burrage, & Lee, JAMA, 329(1), 85-86 (2023); Mortazavi et al., Nature Methods, 5(7), 621-8 (2008); and Peymani, Farzeen, & Prokisch, Pediatr. Investig., 6(1), 29-35 (2022). The technology has several clinical uses such as analyzing the transcriptome of a cancer and determining a type of infection. Huang, Wang, & Yao, Microb. Cell, 8(9), 208-222 (2021). RNA sequencing has also been used to elucidate the pathogenesis of certain diseases and potential treatment approaches. See Huang, Wang, & Yao, Microb. Cell, 8(9), 208-222 (2021). This laboratory technique can detect different transcript isoforms from alternative splicing, chimeric gene fusions, and other genetic changes. Mortazavi et al., Nature Methods, 5(7), 621-8 (2008). With alignment to pathogen organism genomes, comparisons between genetic expression of a pathogen can be made. Fredericks et al., Science Reports, 12(1), 15755 (2022).

Regulatory RNAs regulate metabolic and virulence functions of certain pathogens, showing the increasing pressure to expand the capability of RNA Sequencing to create a full transcriptome in the clinic. See Oliva, Sahr, & Buchrieser, FEMS Microbiol. Rev., 39(3), 331-49 (2015); and Papenfort & Vogel, Front. Cell Infect. Microbiol., 4, 91 (2014). RNA is subject to multiple cellular processes that can affect genetic expression.

Up to 92-94% of human multiexon genes undergo alternative splicing. Houseley & Tollervey, Cell, 136(4), 763-76 (2009). Mutations in RNA modification enzymes were associated with over 100 human diseases. The estimated median mRNA half-life in humans is ten hours, with different functional groups of mRNA decaying at different rates. Yang et al., Genome Res, 2003. 13(8), 1863-72.

The stability of mRNA has also been found to alter gene expression and mRNA life span. RNA viruses evade degradation by maintaining mRNA stability. See Houseley & Tollervey, Cell, 136(4), 763-76 (2009); and Moon, Barnhart, & Wilusz, Curr. Opin. Microbiol., 15(4), 500-5 (2012). Stability of RNA has been measured by the minimum free energy (MFE) of the structure and the ensemble free energy (EFE) of the structure. See, Ding, Chan, & Lawrence, RNA, 11(8), 1157-66 (2005); Doshi et al., BMC Bioinformatics, 5, 105 (2004); Wuchty et al., Biopolymers, 49(2), 145-65 (1999); Lorenz et al., ViennaRNA Package 2.0. Algorithms Mol. Biol., 6, 26 (2011); Trotta, PLOS One, 9(11), e113380 (2014); and Vasudevan& Steitz, Cell, 128(6), 1105-18 (2007).

This EXAMPLE presents an analysis of the stability of RNA-Seq reads from COVID-19 infection patients. The inventors established RNA motifs that either increase or decrease stability of the RNA-Seq read fragment. The inventors also assessed whether the destabilizing RNA motif affects duplicate RNA-Seq reads.

Results. Of the 676 reads from RNA-Seq, there were 137 unique sequences. Thus, 539 reads were identical with another read. Among all the unique read sequences, the average minimum free energy (MFE) in kcal/mol was −30.46 and the average ensemble free energy (EFE) in kcal/mol was −32.94. Of the repeated sequences, there was on sequence that was repeated 328 times. The minimum free energy for this sequence was −33.00 kcal/mol and the ensemble free energy was −35.20 kcal/mol. Despite being highly repetitive, it was only the 48th lowest MFE and the 60th lowest EFE.

For the analysis of the minimum free energy and ensemble free energy values of read sequences in whole gene regions, read sequences were found in six genes, the nucleocapsid (N) gene, the ORF1ab gene, the ORF3a gene, ORF6 gene, ORF8 gene, and the spike (S) gene. The N gene and S gene encode integral structural proteins and the ORF3a, ORF6, and ORF8 genes encode auxiliary genes. The ORF1ab genes encode other nonstructural proteins.

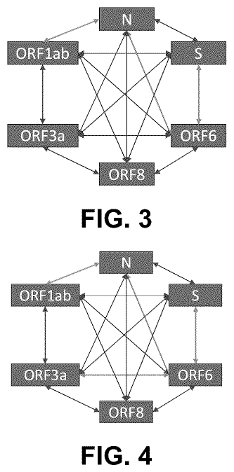

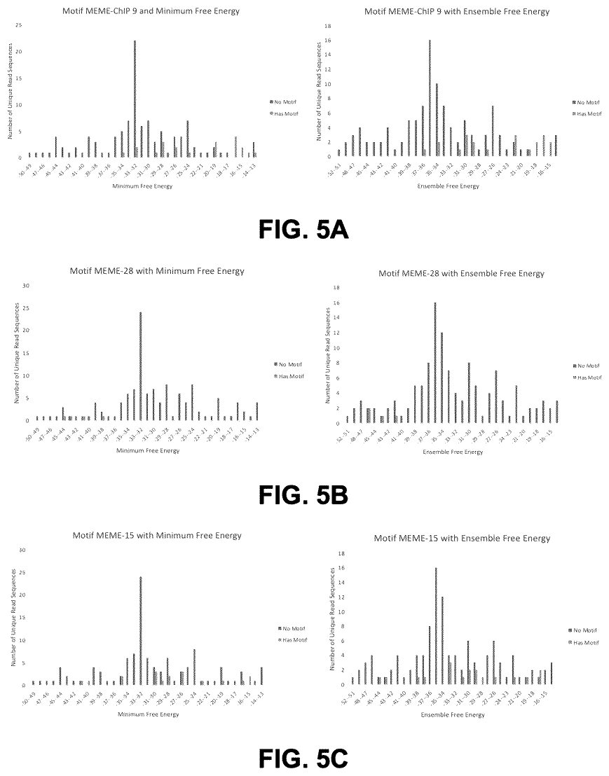

A Welch's ANOVA analysis showed there was at least one mean minimum free energy for a gene that was significantly different from another gene's mean minimum free energy (p=0.0004907). The post hoc analysis assessed fifteen pairs among the six genes and showed three significant relationships. The mean minimum free energy of the N gene was significantly different from that of the ORF1ab gene (p=2.81e-7). The mean MFE of the N gene was also significantly different from that of the ORF6 gene (p=p=0.23). The ORF6 gene's mean minimum free energy differed significantly from the S gene's mean minimum free energy (p=0.037). See schematics in FIG. 3 and FIG. 4,

For ensemble free energy, the Welch's ANOVA analysis showed there was at least one mean ensemble free energy for a gene that differed significantly from another gene's mean ensemble free energy (p=0.002398). The post hoc analysis found four significant pairwise comparisons. The mean ensemble free energy of the N gene differed significantly from that of the ORF1ab gene (p=0.005). The mean ensemble free energy of the N gene was also significantly different from that of the ORF6 gene (p=0.03). The mean ensemble free energy of the ORF3a gene differed significantly from that of the ORF6 gene (p=0.027). The ORF6 gene's mean minimum free energy differed significantly from the S gene's mean ensemble free energy (p=0.036). For the analysis of the minimum free energy and ensemble free energy values within the N gene, a Welch's ANOVA analysis was completed. Later, a Welch's t-test was completed comparing each of the gene groups individually to each other.

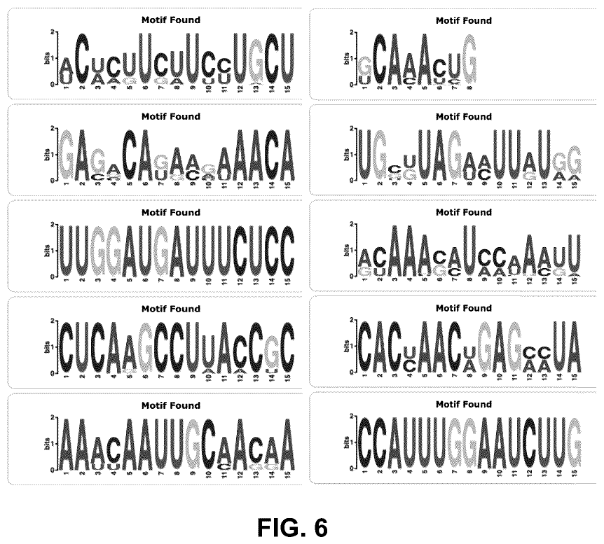

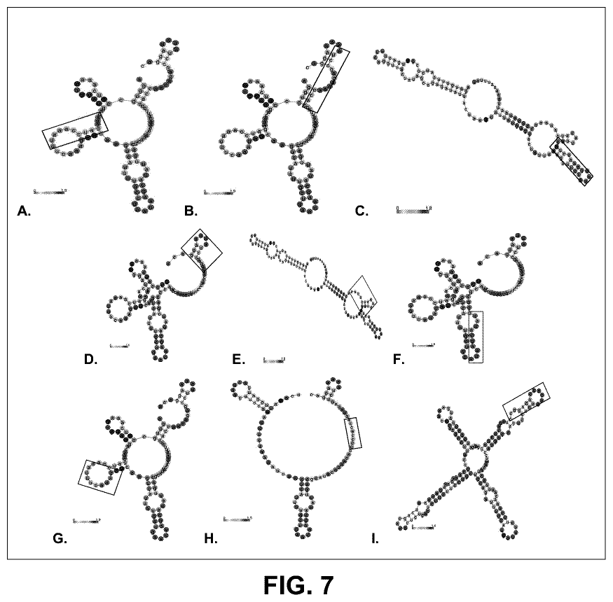

The motif analysis using MEME-ChIP of all the read sequences discovered ten motifs, of which six had known or similar motifs in the database. See sequences in FIG. 6. Three of these motifs have a higher minimum free energy for the read sequences (p=0.00502, p=0.00000422, p=0.00023) and a higher ensemble free energy for the read sequences (p=0.0034, p=0.0000034, p=0.00039). The motif analysis using XSTREME of all the read sequences discovered thirty-four motifs. See FIG. 7. For six of the identified motifs, sequences that had the motifs have significantly different minimum free energy values compared to sequences without the motif (p=0.0275, p=0.0204, p=0.000455, p=0.0082, p=0.00175, p=3.26e-79). These motifs were labeled MEME-9, MEME-10, MEME-21, MEME-22, MEME-27, and MEME-28. For all these motifs besides MEME-10, sequences with the motifs also have significantly different ensemble free energy values compared to sequences without the motif (p=0.0315, p=0.000664, p=0.00695, p=0.00114, p=0.000328). A negative binomial regression was performed to assess if MEME-28, the only motif found to be stabilizing, was associated with sequences with a higher number of duplicates. This analysis was not significant with a p value of 0.266.

Discussion. This initial chi-squared goodness of fit test showed the proportion of reads from different genes were not equal. RNA-Seq does not uniformly collect reads from each gene. There may be factors that influence a particular sequence being detected. One known factor is RNA expression. With different levels of gene expression for different genes, the large number of certain RNA sequences may be greater than other sequences at different time points of a cell.

Degradation and stability may be other factors that play a role in detection by an RNA-Seq assay. When PCR primers are designed based on RNA sequencing data, stability of structure should be included not just in design but also optimization of the work flow.

The results of his EXAMPLE showed the stability of reads from different genes varied. Because genes like ORF6 had sequences that were less stable compared to genes like the S gene, ORF6 may be underrepresented in the RNA-Seq analysis. If true, there will be a significant impact on our interpretation of RNA-Seq results. Genes that may be regarded as lowly expressed and disregarded to focus on seemingly highly expressed genes may be new targets to be reanalyzed. The contributions of these genes to cellular function may have been underestimated.

Within the N gene, reads from different regions also differed in stability. Because all these reads came from the N gene, differences in the quantity of duplicates did not arise from the expression of the N gene itself but the abundance of potentially alternatively spliced RNA and stability of RNA fragments. One sequence in the N gene was heavily duplicated with 328 repeats.

The motif analysis of this EXAMPLE showed motifs that corresponded to destabilization of the RNA and a single motif that corresponded to stabilization of the RNA. These motifs were not present in the most duplicated read sequence, but there may still be motifs in that read sequence that were not detected by our analysis. The most repeated sequence may have motifs that confer increased stability or an increased chance to be detected by RNA-Seq, but it was not discovered.

The motif analysis in this EXAMPLE used both the established motif analysis tool MEME-ChIP and the new motif analysis tool XSTREME. MEME-ChIP is optimized for sequences larger than our average sequence length. MEME-ChIP discovered three of the eight motifs that affected RNA stability that XSTREME could not. These two motif analysis tools discovered different motifs. Both should be used for later assays.

This EXAMPLE provides the discovery of new motifs that may confer increased or decreased stability for RNA. Using the motifs discovered to alter stability while also limiting differing gene expression levels and alternative splicing, the relationship between stability and RNA-Seq read duplications can be further elucidated, allowing for further analysis, and broadening of research implications of the already well-known RNA-Seq experiment.

### Example 6

Deep RNA Sequencing and Aligned Unmapped Reads from Patients to a Custom Genome Constructed and Retrieved from the NCBI Gene Database.

Whole blood samples were collected from patients in the ICU, stored in Paxgene tubes to preserve the integrity of the specimens, and submitted for RNA sequencing by a commercial sequencing service (Azenta/Genewiz).

In this EXAMPLE, the inventors used deep RNA sequencing and aligned unmapped reads from patients to a custom genome constructed and retrieved from the NCBI Gene database. See TABLE 5 in EXAMPLE 1.

All analyses were conducted blinded to clinical data and patient outcomes. Unmapped RNA sequencing reads were aligned to all four custom genomes using the STAR aligner for classification, extraction and count of unmapped reads.

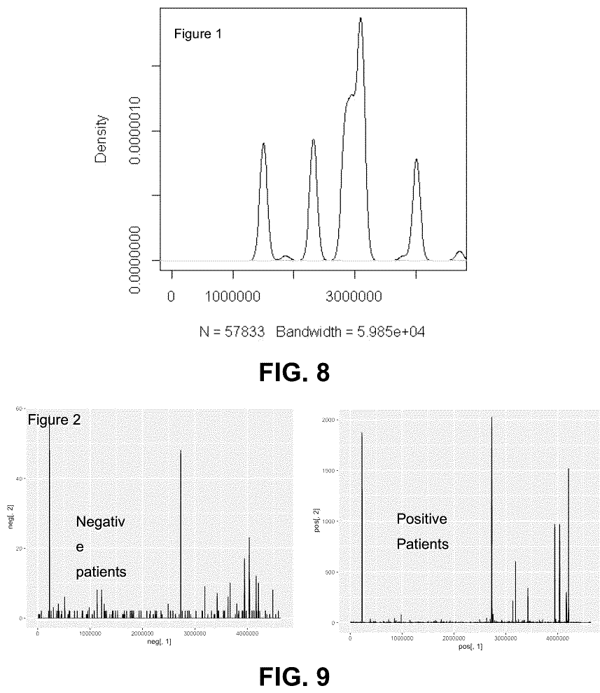

Four custom genomes were created and aligned against the unmapped reads retrieved from the patient. When a read of at least 100 base pairs aligned to the pathogen genome, it was counted as a read a plotted to the pathogen genome. Density plots (see FIG. 8) were then used to identify the areas of the genomes with the most reads. When compared to clinical microbiology data like positive cultures and clinical outcomes like mortality, targets from the pathogen genome were identified as targets to base PCR based tests upon.

Specifically for E. coli. Reads from patients with positive blood cultures were grouped together and compared to reads that occurred from patients that were found to not have an E. coli infection. FIG. 9 shows the reads from the two groups. When comparing there are significantly more reads in the patients with positive E. Coli infections. In addition, using these plots target genes to design PCR tests on clinical microbiology machines.

Specifically for E. coli, the following genes and exact nucleic acid targets will be used based RNA sequencing data. Both ribosomal RNA and mRNA targets will be used. Final targets are blasted against all known genomes to ensure no false signals.
rrIE Ribosomal RNA 50S subunit
Coordinates NC_000913.3:4210165-4210194
IdtD Murein L,D-transpeptidase
Coordinates NC_000913.3:982647-982657
pitB Phosphate Transporter
Coordinates NC_000913.3:3135153-3135165

These three sites were chosen for several reasons including increases in numbers seen in patients with E. coli infections, the target of mRNA since it is known that this last 5-8 minutes and ensuring that the targets are unique only for this pathogen. It is also important to note that identification of these targets with methods such as PCR or nucleic acid probes will identify this as the causative pathogen and change treatment to an appropriate antibiotic.

The work done to identify these targets can be repeated on the other pathogens noted and for all the resistance genes.

### List of Embodiments

Specific compositions and methods of the invention have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as persons skilled in the biomedical art recognizes. When the specification or claims recite method steps or functions in an order, alternative embodiments may perform the functions in a different order or substantially concurrently. The inventive subject matter should not be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by persons of ordinary skill in the biomedical art to which this invention belongs. This invention is not limited to the particular methodology, protocols, reagents, and the like described in this specification and can vary in practice. The terminology used in this specification is not intended to limit the scope of the invention, which is defined solely by the claims.

When a range of values is provided, each intervening value, to the tenth of the unit of the lower limit, unless the context dictates otherwise, between the upper and lower limit of that range and any other stated or intervening value in that range of values.

Some embodiments of the technology described can be defined according to the following numbered paragraphs:
1. A direct from blood, without the need for culture, reverse transcriptase polymerase chain reaction (RT-qPCR) test for bacteria causing bacteremia, based on the RNA identified in patients with bacteremia caused by these bacteria, optionally selected from the group consisting of S. aureus, P. aeruginosa, and H. influenzae.
2. A method to validate these RT-qPCR tests in samples from patients with and without bacteremia.
3. A direct from blood, without the need for culture, reverse transcriptase polymerase chain reaction (RT-qPCR) test for bacteria causing pneumonia, based on the RNA identified in patients with pneumonia caused by these bacteria, optionally selected from the group consisting of S. aureus, P. aeruginosa, and H. influenzae.
4. A method to validate these RT-qPCR tests in samples from patients with and without pneumonia.
5. An RT-PCR for the most common resistance genes expressed that would influence treatment of bacteria, optionally selected from the group consisting of S. aureus, P. aeruginosa, and H. influenzae, using the RNA from patients with infections.
6. A method to validate these PCR tests for resistance genes in samples from patients with and without infections.

### REFERENCES

Persons having ordinary skill in the biomedical art can rely on the following patents, patent applications, scientific books, and scientific publications for enabling methods:

### Patent Citations

Intl. Pat. Pub. WO2021163692A1 (Rhode Island Hospital), RNA sequencing to diagnose sepsis, published Aug. 19, 2021.

### Nonpatent Citations

Al-Hasan, Winders, Bookstaver, & Justo, Direct measurement of performance: A new era in antimicrobial stewardship. Antibiotics (Basel, Switzerland). 8(3) (2019).
Bajaj, Singh, & Virdi, Escherichia coli B-lactamases: What really matters. Frontiers in Microbiology, 7, 417 (2016).
Blackberg et al., Time to blood culture positivity: An independent predictor of mortality in Streptococcus pyogenes bacteremia. Open Forum Infectious Diseases, 9(6), ofac163 (2022).
Blenkiron et al., Uropathogenic Escherichia coli releases extracellular vesicles that are associated with RNA. PLOS One, 11(8), e0160440 (2016).
Bonine et al., Impact of delayed appropriate antibiotic therapy on patient outcomes by antibiotic resistance status from serious gram-negative bacterial infections. The American Journal of the Medical Sciences, 357(2), 103-10 (2019).
Bortolaia et al., ResFinder 4.0 for predictions of phenotypes from genotypes. The Journal of Antimicrobial Chemotherapy, 75(12), 3491-500 (2020).
Bustin & Huggett, qPCR primer design revisited. Biomolecular detection and quantification, 14, 19-28 (2017).
Bustin, Mueller, & Nolan, Parameters for successful PCR primer design. Methods in Molecular Biology (Clifton, NJ), 2065, 5-22 (2020).
Camargo et al., Next-generation sequencing of microbial cell-free DNA for rapid noninvasive diagnosis of infectious diseases in immunocompromised hosts. F1000Research, 8, 1194 (2019).
Cascella et al., Features, Evaluation, and Treatment of Coronavirus (COVID-19), in StatPearls (Treasure Island, Florida, 2023).
Chambers & Deleo, Waves of resistance: Staphylococcus aureus in the antibiotic era. Nature Reviews Microbiology, (9), 629-41 (2009).
Chambers, Methicillin resistance in staphylococci: molecular and biochemical basis and clinical implications. Clinical Microbiology Reviews, 10(4), 781-91 (1997).
Cheng et al., Contribution of coagulases towards Staphylococcus aureus disease and protective immunity. PLOS Pathogens, 6(8), e1001036 (2010).
Clatworthy, Pierson, & Hung, Targeting virulence: A new paradigm for antimicrobial therapy. Nature Chemical Biology; 3(9), 541-8 (2007).
Covert, Bashore, Edds, & Lewis, Utility of the respiratory viral panel as an antimicrobial stewardship tool. Journal of Clinical Pharmacy and Therapeutics, 46(2), 277-85 (2021).
D'Mello et al., An in vivo atlas of host-pathogen transcriptomes during Streptococcus pneumoniae colonization and disease. Proceedings of the National Academy of Sciences, 117:33507-18 (2020).
De Bus et al., Antimicrobial de-escalation in the critically ill patient and assessment of clinical cure: the DIANA study. Intensive Care Medicine, 46(7), 1404-17 (2020).
Dersch, Khan, Mühlen, & Görke, Roles of regulatory RNAs for antibiotic resistance in bacteria and their potential value as novel drug targets. Frontiers in Microbiology, 8, 803 (2017).
Deschamps-Francoeur, Simoneau, & Scott, Handling multi-mapped reads in RNA-seq., Comput. Struct. Biotechnol. J., 18, 1569-1576 (2020).
Ding, Chan, & Lawrence, RNA secondary structure prediction by centroids in a Boltzmann weighted ensemble. RNA, 11(8), 1157-66 (2005).
Dingle et al., Prevalence and characterization of the cefazolin inoculum effect in North American methicillin-susceptible Staphylococcus aureus isolates. Journal of Clinical Microbiology, e0249521 (2022).
Dobin et al., STAR: Ultrafast universal RNA-seq aligner. Bioinformatics (Oxford, England), 29(1), 15-21 (2013).
Doshi et al., Evaluation of the suitability of free-energy minimization using nearest-neighbor energy parameters for RNA secondary structure prediction. BMC Bioinformatics, 5, 105 (2004).
Duncan, Youngstein, Kirrane, & Lonsdale, Diagnostic challenges in sepsis. Current Infectious Disease Reports, 23(12), 22 (2021).
Easterday, Van Ert, Zanecki, & Keim, Specific detection of Bacillus anthracis using a TaqMan mismatch amplification mutation assay. BioTechniques, 38(5), 731-5 (2005).
Eizenga et al., Pangenome graphs. Annual Review of Genomics and Human Genetics, 21, 139-62 (2020).
El-Sadr, Vasan, & El-Mohandes, Facing the new Covid-19 reality. N. Engl. J. Med., 388(5), 385-387 (2023).
Evans et al., Surviving Sepsis Campaign: International guidelines for management of sepsis and septic shock 2021. Critical Care Medicine, 49(11), e1063-e143 (2021).
Fajnzylber et al., SARS-COV-2 viral load is associated with increased disease severity and mortality. Nature Commun, 11(1), 5493 (2020).
Ferguson et al., The Berlin definition of ARDS: An expanded rationale, justification, and supplementary material. Intensive Care Medicine, 8(10), 1573-82 (2012).
Fredericks et al., Deep RNA sequencing of intensive care unit patients with COVID-19, Scientific Reports, 12(1) (January 2022).
Fredericks et al., Deep RNA sequencing of intensive care unit patients with COVID-19. Sci. Rep., 12(1), 15755 (2022).
Fu et al., Elimination of PCR duplicates in RNA-seq and small RNA-seq using unique molecular identifiers. BMC Genomics, 19(1), 531 (2018).
Glaab & Skopek, A novel assay for allelic discrimination that combines the fluorogenic 5′ nuclease polymerase chain reaction (TaqMan) and mismatch amplification mutation assay. Mutation Research, 430(1), 1-12 (1999).
Global burden of bacterial antimicrobial resistance in 2019: A systematic analysis. Lancet (London, England), 399(10325), 629-55 (2022).
Guo et al., Prevalence and therapies of antibiotic-resistance in Staphylococcus aureus. Frontiers in Cellular and Infection Microbiology, 10, 107 (2020).
Gutmann, Williamson, Collatz, & Acar, Mechanisms of beta-lactam resistance in Hæemophilus influenzae. European Journal of Clinical Microbiology & Infectious Diseases: Official publication of the European Society of Clinical Microbiology, 7(5), 610-5 (1988).
Harbottle, Thakur, Zhao, & White, Genetics of antimicrobial resistance. Animal Biotechnology, 17(2), 111-24 (2006).
Hashim, Mabrouk, & Al-Atabany, Review of different sequence motif finding algorithms. Avicenna J. Med. Biotechnol., 11(2), 130-148 (April-June 2019).
Heinrich et al., SARS-COV-2 blood RNA load predicts outcome in critically ill COVID-19 patients. Open Forum Infect. Dis., 8(11), ofab509 (2021).
Houseley & Tollervey, The many pathways of RNA degradation. Cell, 136(4), 763-76 (2009).
Huang, Wang, & Yao, Understanding the pathogenesis of infectious diseases by single-cell RNA sequencing. Microb. Cell, 8(9), 208-222 (2021).
Hui, DelMonte, & Ranade, Genotyping using the TaqMan assay. Current Protocols in Human Genetics, Chapter 2:Unit 2.10 (2008).
Islam et al., Chromosomal mechanisms of aminoglycoside resistance in Pseudomonas aeruginosa isolates from cystic fibrosis patients. Clinical Microbiology and Infection: Official publication of the European Society of Clinical Microbiology and Infectious Diseases, 15(1), 60-6 (2009).
Jacobs et al., Severe acute respiratory syndrome coronavirus 2 viremia is associated with coronavirus disease 2019 severity and predicts clinical outcomes. Clin. Infect. Dis., 74(9), 1525-1533 (2022).
Jacoby, AmpC beta-lactamases. Clinical Microbiology Reviews. 22(1), 161-82 (2009).
Karczmarczyk et al., Mechanisms of fluoroquinolone resistance in Escherichia coli isolates from food-producing animals. Applied and Environmental Microbiology, 77(20), 7113-20 (2011).
Kassakian & Mermel, Changing epidemiology of infections due to extended spectrum beta-lactamase producing bacteria. Antimicrobial Resistance and Infection Control, 3(1), 9 (2014).
Ketkar, Burrage, & Lee, RNA Sequencing as a Diagnostic Tool. JAMA, 329(1), 85-86 (2023).
Kumar et al., Duration of hypotension before initiation of effective antimicrobial therapy is the critical determinant of survival in human septic shock. Critical Care Medicine, 34(6), 1589-96 (2006).
Langelier et al., Detection of pneumonia pathogens from plasma cell-free DNA. American Journal Of Respiratory and Critical Care Medicine, 201(4), 491-5 (2020).
Livak, Allelic discrimination using fluorogenic probes and the 5′ nuclease assay. Genetic Analysis: Biomolecular Engineering, 14(5-6), 143-9 (1999).
Lorenz et al., ViennaRNA Package 2.0. Algorithms Mol Biol, 6, 26 (2011).
Lu et al., US CDC real-time reverse transcription PCR panel for detection of severe acute respiratory syndrome coronavirus 2. Emerg. Infect. Dis., 26(8), 1654-65 (2020).
Mangul et al., ROP: dumpster diving in RNA-sequencing to find the source of 1 trillion reads across diverse adult human tissues. Genome Biology, 19(1), 36 (2018).
Marini et al., AMR-meta: A k-mer and metafeature approach to classify antimicrobial resistance from high-throughput short-read metagenomics data. GigaScience, 11 (2022).
McCaskill, The equilibrium partition function and base pair binding probabilities for RNA secondary structure. Biopolymers, 29(6-7), 1105-19 (1990).
Mei-Sheng, Riley & Olans, Implementing an antimicrobial stewardship program in the intensive care unit by engaging critical care nurses. Critical Care Nursing Clinics of North America, 33(4), 369-80 (2021).
Modi & Kovacs, Hospital-acquired and ventilator-associated pneumonia: Diagnosis, management, and prevention. Cleveland Clinic Journal of Medicine, 87(10), 633-9 (2020).
Monaghan et al., Deep RNA sequencing of intensive care unit patients with COVID-19. medRxiv (2021).
Moon, Barnhart, & Wilusz, Inhibition and avoidance of mRNA degradation by RNA viruses. Curr. Opin. Microbiol., 15(4), 500-5 (2012).
Mortazavi et al., Mapping and quantifying mammalian transcriptomes by RNA-Seq. Nature Methods, 5(7), 621-8 (2008).
Noureen, Tada, Kawashima, & Arita, Rearrangement analysis of multiple bacterial genomes. BMC Bioinformatics, 20(Suppl 23), 631 (2019).
Oliva, Sahr, & Buchrieser, Small RNAs, 5′ UTR elements and RNA-binding proteins in intracellular bacteria: impact on metabolism and virulence. FEMS Microbiol. Rev., 39(3), 331-49 (2015).
Oliva, Sahr, & Buchrieser, Small RNAs, 5′ UTR elements and RNA-binding proteins in intracellular bacteria: impact on metabolism and virulence. FEMS Microbiol. Rev., 39(3), 331-49 (2015).
Opota, Jaton, & Greub, Microbial diagnosis of bloodstream infection: towards molecular diagnosis directly from blood. Clinical Microbiology and Infection: the official publication of the European Society of Clinical Microbiology and Infectious Diseases, 21(4), 323-31 (2015).
Palavecino, Rapid methods for detection of MRSA in clinical specimens. Methods in Molecular Biology (Clifton, NJ), 2069, 29-45 (2020).
Papenfort & Vogel, Small RNA functions in carbon metabolism and virulence of enteric pathogens. Front Cell Infect Microbiol, 4, 91 (2014).
Paterson et al., Outcome of cephalosporin treatment for serious infections due to apparently susceptible organisms producing extended-spectrum beta-lactamases: implications for the clinical microbiology laboratory. Journal of Clinical Microbiology, 39(6), 2206-12 (2001).
Peeling, Heymann, Teo, & Garcia, Diagnostics for COVID-19: Moving from pandemic response to control. Lancet, 399(10326), 757-768 (2022).
Peymani, Farzeen, & Prokisch, RNA sequencing role and application in clinical diagnostic. Pediatr Investig, 6(1), 29-35 (2022).
Rhee et al., Prevalence of antibiotic-resistant pathogens in culture-proven sepsis and outcomes associated with inadequate and broad-spectrum empiric antibiotic use. JAMA Network Open, 3(4), e202899 (2020).
Rivera-Izquierdo et al., OXA-48 carbapenemase-producing enterobacterales in Spanish hospitals: An updated comprehensive review on a rising antimicrobial resistance. Antibiotics (Basel, Switzerland), 10(1) (2021)
Rodriguez-Serrano et al., Detection of SARS-COV-2 RNA in serum is associated with increased mortality risk in hospitalized COVID-19 patients. Sci. Rep., 11(1), 13134 (2021).
Rudd et al., Global, regional, and national sepsis incidence and mortality, 1990-2017: Analysis for the Global Burden of Disease Study. Lancet (London, England), 395(10219), 200-11 (2020).
Rupp & Fey, Extended spectrum beta-lactamase (ESBL)-producing Enterobacteriaceae: Considerations for diagnosis, prevention and drug treatment. Drugs, 63(4), 353-65 (2003).
Salah et al., Distribution of quinolone resistance gene (qnr) in ESBL-producing Escherichia coli and Klebsiella spp. in Lomé, Togo. Antimicrobial Resistance and Infection Control, 8, 104 (2019).
Seok, Jeon, & Park, Antimicrobial therapy and antimicrobial stewardship in sepsis. Infection & Chemotherapy, 52(1), 19-30 (2020).
Shankar-Hari et al., Developing a new definition and assessing new clinical criteria for septic shock: For the Third International Consensus Definitions for Sepsis and Septic Shock (Sepsis-3). JAMA, 315(8), 775-87 (2016).
Sidstedt et al., Inhibition mechanisms of hemoglobin, immunoglobulin G, and whole blood in digital and real-time PCR. Analytical and Bioanalytical Chemistry, 410(10), 2569-83 (2018).
Sterne-Weiler et al., Efficient and accurate quantitative profiling of alternative splicing patterns of any complexity on a laptop. Molecular Cell, 72(1), 187-200.e6 (2018).
Strich, Heil, & Masur, Considerations for empiric antimicrobial therapy in sepsis and septic shock in an era of antimicrobial resistance. The Journal of Infectious Diseases, 222(Suppl 2), S119-s31 (2020).
Suzuki, Horinouchi, & Furusawa, Prediction of antibiotic resistance by gene expression profiles. Nature Communications, 5, 5792 (2014).
Tada, Tanizawa, & Arita, Visualization of consensus genome structure without using a reference genome. BMC genomics, 18(Suppl 2), 208 (2017).
Taniguchi et al., Quantifying E. coli proteome and transcriptome with single-molecule sensitivity in single cells. Science (New York, NY), 329(5991), 533-8 (2010).
Tchesnokova et al., Pandemic fluoroquinolone resistant Escherichia coli clone ST1193 emerged via simultaneous homologous recombinations in 11 gene loci. Proceedings of the National Academy of Sciences of the United States of America, 116(29), 14740-8 (2019).
Teixeira et al., Aminoglycoside resistance genes in Pseudomonas aeruginosa Isolates from Cumana, Venezuela. Revista do Instituto de Medicina Tropical de Sao Paulo, 58, 13 (2016).
Teshome et al., Duration of exposure to antipseudomonal β-lactam antibiotics in the critically ill and development of new resistance. Pharmacotherapy, 39(3), 261-70 (2019).
Tindal et al., Emerging therapeutic targets for sepsis, Expert Opinion on Therapeutic Targets, 25(3), 175-189 (Mar. 4, 2021.)
Tristram, Jacobs, & Appelbaum, Antimicrobial resistance in Haemophilus influenzae. Clinical Microbiology Reviews, 20(2), 368-89 (2007).
Trotta, On the normalization of the minimum free energy of RNAs by sequence length. PLOS One, 9(11), e113380 (2014).
Vasudevan & Steitz, AU-rich-element-mediated upregulation of translation by FXR1 and Argonaute 2. Cell, 128(6), 1105-18 (2007).
Vazquez-Guillamet et al., Using the number needed to treat to assess appropriate antimicrobial therapy as a determinant of outcome in severe sepsis and septic shock. Critical Care Medicine, 42(11), 2342-9 (2014).
Wang et al., Pseudomonas aeruginosa detection using conventional PCR and quantitative real-time PCR Based on species-specific novel gene targets identified by pangenome analysis. Frontiers in Microbiology, 13, 820431 (2022).
Webb et al., Broad-spectrum antibiotic use and poor outcomes in community-onset pneumonia: a cohort study. The European Respiratory Journal, 54(1) (2019).
Whole-genome sequencing as part of national and international surveillance programmes for antimicrobial resistance: a roadmap. BMJ Global Health, 5(11) (2020).
Wood, Lu, & Langmead, Improved metagenomic analysis with Kraken2. Genome Biology, 20(1), 257 (2019).
Wu et al., The SARS-COV-2 nucleocapsid protein: its role in the viral life cycle, structure and functions, and use as a potential target in the development of vaccines and diagnostics. Virology Journal, 20(1), 6 (2023).
Wuchty et al., Complete suboptimal folding of RNA and the stability of secondary structures. Biopolymers, 49(2), 145-65 (1999).
Yan, Chang, & Wang, Laboratory testing of SARS-COV, MERS-COV, and SARS-COV-2 (2019-nCOV): Current status, challenges, and countermeasures. Reviews in Medical Virology, 30(3), e2106 (2020).
Yang et al., Decay rates of human mRNAs: Correlation with functional characteristics and sequence attributes. Genome Res, 13(8), 1863-72 (2003).
Yang et al., Prevalence and fluoroquinolone resistance of Pseudomonas aeruginosa in a hospital of South China. International Journal of Clinical and Experimental Medicine, 8(1), 1386-90 (2015).
Yu et al., Low prevalence of bloodstream infection and high blood culture contamination rates in patients with COVID-19. PLOS One, 15(11), e0242533 (2020).
Zhang, Yang, & Makam, Utility of blood cultures in pneumonia. The American Journal of Medicine, 132(10), 1233-8 (2019).
Zhou et al., Single-cell RNA sequencing reveals the immune microenvironment and signaling networks in cystitis glandularis. Frontiers in Immunology, 14 (2023).
Zuker & Stiegler, Optimal computer folding of large RNA sequences using thermodynamics and auxiliary information. Nucleic Acids Res, 9(1), 133-48 (1981).

### Textbooks and Technical References

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al., (eds.), published by Blackwell Science Ltd., 1999-2012 (ISBN 9783527600908).
The Merck Manual of Diagnosis and Therapy, 19^{th} edition (Merck Sharp & Dohme Corp., 2018).
Pharmaceutical Sciences 23^{rd} edition (Elsevier, 2020).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

1. A direct from blood, without culture, reverse transcriptase polymerase chain reaction (RT-qPCR) test for bacteria causing bacteremia, based on the RNA identified in patients with bacteremia caused by these bacteria

2. The RT-qPCR test of claim 1, wherein the bacteria causing bacteremia are selected from the group consisting of Staphylococcus aureus, Escherichia coli, and Haemophilus influenzae.

3. A direct from blood, without culture, reverse transcriptase polymerase chain reaction (RT-qPCR) test for bacteria causing pneumonia, based on the RNA identified in patients with pneumonia caused by these bacteria.

4. The RT-qPCR test of claim 3, wherein the bacteria causing pneumonia are selected from the group consisting of Staphylococcus aureus, Pseudomonas aeruginosa, and Haemophilus influenzae.

5. A direct from blood, without culture, reverse transcriptase polymerase chain reaction (RT-qPCR) test for the most common resistance genes expressed that would influence treatment of bacteria.

6. The PCR test of claim 5, wherein the most common resistance genes expressed that would influence treatment of bacteria selected from the group consisting of Staphylococcus aureus, Pseudomonas aeruginosa, and Haemophilus influenzae, using the RNA from patients with infections.
